SEQUENCE LISTING (73 sequences)

SEQ ID NO: 1 - RNA, 37 nt, synthetic construct
  misc_feature 1..37: note = ACH siRNA
  misc_difference 19: note = n is selected from the nucleotides A and C
  source 1..37: mol_type = other RNA
gcaggcatcctcagctacngggtgggcttcttcctgt

SEQ ID NO: 2 - RNA, 19 nt, synthetic construct
  misc_feature 1..19: note = ACH siRNA
  misc_difference 1: note = N is selected from the group consisting of A, G, C and U
  misc_difference 2: note = N is selected from the group consisting of A, G, C and U
  misc_difference 5: note = N is selected from the group consisting of A, G, C and U
  misc_difference 6: note = N is selected from the group consisting of A and C
  misc_difference 13: note = N is selected from the group consisting of A, G, C and U
  misc_difference 15: note = N is selected from the group consisting of A, G, C and U
  source 1..19: mol_type = other RNA
nntanngggtggncntctt

SEQ ID NO: 3 - RNA, 19 nt, synthetic construct
  misc_feature 1..19: note = ACH siRNA
  source 1..19: mol_type = other RNA
gctatagggtgggcttctt

SEQ ID NO: 4 - RNA, 19 nt, synthetic construct
  misc_feature 1..19: note = ACH siRNA
  source 1..19: mol_type = other RNA
gctacagggtgggcatctt

SEQ ID NO: 5 - RNA, 19 nt, synthetic construct
  misc_feature 1..19: note = ACH siRNA
  misc_difference 1: note = N is selected from the group consisting of A and C
  misc_difference 2: note = N is selected from the group consisting of A, G, C and U
  misc_difference 3: note = N is selected from the group consisting of A, G, C and U
  misc_difference 13: note = N is selected from the group consisting of A, G, C and U
  misc_difference 15: note = N is selected from the group consisting of A, G, C and U
  source 1..19: mol_type = other RNA
nnngtgggcttcntnctgt

SEQ ID NO: 6 - RNA, 19 nt, synthetic construct
  misc_feature 1..19: note = ACH siRNA
  source 1..19: mol_type = other RNA
aaggtgggcttcttcctgt

SEQ ID NO: 7 - RNA, 19 nt, synthetic construct
  misc_feature 1..19: note = ACH siRNA
  source 1..19: mol_type = other RNA
agggtgggcttctttctgt

SEQ ID NO: 8 - RNA, 19 nt, synthetic construct
  misc_feature 1..19: note = ACH siRNA
  misc_difference 1: note = N is selected from the group consisting of A, G, C and U
  misc_difference 8: note = N is selected from the group consisting of A, G, C and U
  misc_difference 9: note = N is selected from the group consisting of A and C
  misc_difference 10: note = N is selected from the group consisting of A, G, C and U
  misc_difference 13: note = N is selected from the group consisting of A, G, C and U
  misc_difference 14: note = N is selected from the group consisting of A, G, C and U
  source 1..19: mol_type = other RNA
ncagctannnggnnggctt

SEQ ID NO: 9 - RNA, 19 nt, synthetic construct
  misc_feature 1..19: note = ACH siRNA
  source 1..19: mol_type = other RNA
tcagctacagggtcggctt

SEQ ID NO: 10 - RNA, 19 nt, synthetic construct
  misc_feature 1..19: note = ACH siRNA
  source 1..19: mol_type = other RNA
tcagctacagggagggctt

SEQ ID NO: 11 - RNA, 19 nt, synthetic construct
  misc_feature 1..19: note = ACH siRNA
  misc_difference 1: note = N is selected from the group consisting of A, G, C and U
  misc_difference 12: note = N is selected from the group consisting of A, G, C and U
  misc_difference 13: note = N is selected from the group consisting of A, G, C and U
  misc_difference 14: note = N is selected from the group consisting of A, G, C and U
  misc_difference 15: note = N is selected from the group consisting of A and C
  misc_difference 16: note = N is selected from the group consisting of A, G, C and U
  misc_difference 18: note = N is selected from the group consisting of A, G, C and U
  source 1..19: mol_type = other RNA
ncatcctcagcnnnnngnt

SEQ ID NO: 12 - RNA, 19 nt, synthetic construct
  misc_feature 1..19: note = ACH siRNA
  source 1..19: mol_type = other RNA
gcatcctcagctatagggt

SEQ ID NO: 13 - RNA, 19 nt, synthetic construct
  misc_feature 1..19: note = ACH siRNA
  misc_difference 2: note = N is selected from the group consisting of A, G, C and U
  misc_difference 6: note = N is selected from the group consisting of A, G, C and U
  misc_difference 13: note = N is selected from the group consisting of A, G, C and U
  misc_difference 15: note = N is selected from the group consisting of A, G, C and U
  misc_difference 18: note = N is selected from the group consisting of A, G, C and U
  misc_difference 19: note = N is selected from the group consisting of A and C
  source 1..19: mol_type = other RNA
gnaggnatcctcngntann

SEQ ID NO: 14 - RNA, 37 nt, synthetic construct
  misc_feature 1..37: note = ACH siRNA
  misc_difference 2: note = N is selected from C and U
  misc_difference 5: note = N is selected from G and U
  misc_difference 6: note = N is selected from C and U
  misc_difference 11: note = N is selected from A and U
  misc_difference 13: note = N is selected from A and U
  misc_difference 14: note = N is selected from G and U
  misc_difference 15: note = N is selected from C, G and U
  misc_difference 17: note = N is selected from A and U
  misc_difference 18: note = N is selected from C and U
  misc_difference 19: note = N is selected from the nucleotides A and C
  misc_difference 20: note = N is selected from G, A and U
  misc_difference 21: note = N is selected from G and U
  misc_difference 22: note = N is selected from G and A
  misc_difference 23: note = N is selected from A and U
  misc_difference 24: note = N is selected from G and C
  misc_difference 26: note = N is selected from G and C
  misc_difference 28: note = N is selected from A and U
  misc_difference 31: note = N is selected from A and U
  misc_difference 33: note = N is selected from C and U
  source 1..37: mol_type = other RNA
gnagnnatccncnnntnnnnnnnngncntcntnctgt

SEQ ID NO: 15 - RNA, 19 nt, synthetic construct
  misc_feature 1..19: note = ACH siRNA
  source 1..19: mol_type = other RNA
gcaggcatcctcagctaca

SEQ ID NO: 16 - RNA, 19 nt, synthetic construct
  misc_feature 1..19: note = ACH siRNA
  source 1..19: mol_type = other RNA
gcaggcatcctcagctata

SEQ ID NO: 17 - RNA, 19 nt, synthetic construct
  misc_feature 1..19: note = ACH siRNA
  source 1..19: mol_type = other RNA
gcaggcatcctcaggtaca

SEQ ID NO: 18 - RNA, 19 nt, synthetic construct
  misc_feature 1..19: note = ACH siRNA
  source 1..19: mol_type = other RNA
gcaggcatcctctgctaca

SEQ ID NO: 19 - RNA, 19 nt, synthetic construct
  misc_feature 1..19: note = ACH siRNA
  source 1..19: mol_type = other RNA
gctacagggtgggcttctt

SEQ ID NO: 20 - RNA, 19 nt, synthetic construct
  misc_feature 1..19: note = ACH siRNA
  source 1..19: mol_type = other RNA
gctacagggtggccttctt

SEQ ID NO: 21 - RNA, 19 nt, synthetic construct
  misc_feature 1..19: note = ACH siRNA
  source 1..19: mol_type = other RNA
agggtgggcttcttcctgt

SEQ ID NO: 22 - RNA, 19 nt, synthetic construct
  misc_feature 1..19: note = ACH siRNA
  source 1..19: mol_type = other RNA
agggtgggcttcatcctgt

SEQ ID NO: 23 - RNA, 19 nt, synthetic construct
  misc_feature 1..19: note = ACH siRNA
  source 1..19: mol_type = other RNA
tcagctacagggtgggctt

SEQ ID NO: 24 - RNA, 19 nt, synthetic construct
  misc_feature 1..19: note = ACH siRNA
  source 1..19: mol_type = other RNA
tcagctatagggtgggctt

SEQ ID NO: 25 - RNA, 19 nt, synthetic construct
  misc_feature 1..19: note = ACH siRNA
  source 1..19: mol_type = other RNA
gcatcctcagctacagggt

SEQ ID NO: 26 - RNA, 19 nt, synthetic construct
  misc_feature 1..19: note = ACH siRNA
  source 1..19: mol_type = other RNA
gcatcctcagcttcagggt

SEQ ID NO: 27 - RNA, 19 nt, synthetic construct
  misc_feature 1..19: note = ACH siRNA
  source 1..19: mol_type = other RNA
gcatcctcagccacagggt

SEQ ID NO: 28 - RNA, 19 nt, synthetic construct
  misc_feature 1..19: note = ACH siRNA
  source 1..19: mol_type = other RNA
gtaggcatcctcagctaca

SEQ ID NO: 29 - RNA, 19 nt, synthetic construct
  misc_feature 1..19: note = ACH siRNA
  source 1..19: mol_type = other RNA
gcaggtatcctcagctaca

SEQ ID NO: 30 - RNA, 19 nt, synthetic construct
  misc_feature 1..19: note = ACH siRNA
  source 1..19: mol_type = other RNA
gttacagggtgggcttctt

SEQ ID NO: 31 - RNA, 19 nt, synthetic construct
  misc_feature 1..19: note = ACH siRNA
  source 1..19: mol_type = other RNA
tctacagggtgggcttctt

SEQ ID NO: 32 - RNA, 19 nt, synthetic construct
  misc_feature 1..19: note = ACH siRNA
  source 1..19: mol_type = other RNA
agtgtgggcttcttcctgt

SEQ ID NO: 33 - RNA, 19 nt, synthetic construct
  misc_feature 1..19: note = ACH siRNA
  source 1..19: mol_type = other RNA
tcatcctcagctacagggt

SEQ ID NO: 34 - RNA, 19 nt, synthetic construct
  misc_feature 1..19: note = ACH siRNA
  source 1..19: mol_type = other RNA
gcatcctcagctacatggt

SEQ ID NO: 35 - RNA, 19 nt, synthetic construct
  misc_feature 1..19: note = ACH siRNA
  source 1..19: mol_type = other RNA
gcatcctcagctacaggat

SEQ ID NO: 36 - RNA, 19 nt, synthetic construct
  misc_feature 1..19: note = ACH siRNA
  source 1..19: mol_type = other RNA
acagctacagggtgggctt

SEQ ID NO: 37 - RNA, 19 nt, synthetic construct
  misc_feature 1..19: note = ACH siRNA
  source 1..19: mol_type = other RNA
tcagctacatggtgggctt

SEQ ID NO: 38 - DNA, 17 bp, synthetic construct
  misc_feature 1..17: note = G380R_Primer pair 1 forward
  source 1..17: mol_type = other DNA
aggcatcctcagctaca

SEQ ID NO: 39 - DNA, 20 bp, synthetic construct
  misc_feature 1..20: note = G380R_Primer pair 1 reverse
  source 1..20: mol_type = other DNA
ggcaggcagctcgagctcgg

SEQ ID NO: 40 - DNA, 20 bp, synthetic construct
  misc_feature 1..20: note = G380R_Primer pair 2 forward
  source 1..20: mol_type = other DNA
ctacagggtgggcttcttcc

SEQ ID NO: 41 - DNA, 20 bp, synthetic construct
  misc_feature 1..20: note = G380R_Primer pair 2 reverse
  source 1..20: mol_type = other DNA
gtggtgtgttggagctcatg

SEQ ID NO: 42 - DNA, 20 bp, synthetic construct
  misc_feature 1..20: note = G380R_Primer pair 3 forward
  source 1..20: mol_type = other DNA
caggcatcctcagctacagg

SEQ ID NO: 43 - DNA, 18 bp, synthetic construct
  misc_feature 1..18: note = G380R_Primer pair 3 reverse
  source 1..18: mol_type = other DNA
tcatggacgcgttggact

SEQ ID NO: 44 - DNA, 29 bp, synthetic construct
  source 1..29: mol_type = other DNA
aataaatcataaaggcatcctcagctaca

SEQ ID NO: 45 - DNA, 32 bp, synthetic construct
  source 1..32: mol_type = other DNA
aataaatcataaggcaggcagctcgagctcgg

SEQ ID NO: 46 - DNA, 17 bp, synthetic construct
  source 1..17: mol_type = other DNA
ggcatcctcagctacag

SEQ ID NO: 47 - DNA, 18 bp, synthetic construct
  source 1..18: mol_type = other DNA
caggcatcctcagctaca

SEQ ID NO: 48 - DNA, 17 bp, synthetic construct
  source 1..17: mol_type = other DNA
cgagacagctcccattt

SEQ ID NO: 49 - DNA, 17 bp, synthetic construct
  source 1..17: mol_type = other DNA
tctctccttgcacaacg

SEQ ID NO: 50 - DNA, 18 bp, synthetic construct
  source 1..18: mol_type = other DNA
tgtagctgaggatgcctg

SEQ ID NO: 51 - DNA, 17 bp, synthetic construct
  source 1..17: mol_type = other DNA
tgtagctgaggatgcct

SEQ ID NO: 52 - DNA, 18 bp, synthetic construct
  source 1..18: mol_type = other DNA
catcactctgcgtggctg

SEQ ID NO: 53 - DNA, 19 bp, synthetic construct
  source 1..19: mol_type = other DNA
tgtagctgaggatgcctgc

SEQ ID NO: 54 - DNA, 29 bp, synthetic construct
  source 1..29: mol_type = other DNA
aataaatcataaggcatcctcagctacag

SEQ ID NO: 55 - DNA, 30 bp, synthetic construct
  source 1..30: mol_type = other DNA
aataaatcataacaggcatcctcagctaca

SEQ ID NO: 56 - DNA, 29 bp, synthetic construct
  source 1..29: mol_type = other DNA
aataaatcataacgagacagctcccattt

SEQ ID NO: 57 - DNA, 29 bp, synthetic construct
  source 1..29: mol_type = other DNA
aataaatcataatctctccttgcacaacg

SEQ ID NO: 58 - DNA, 30 bp, synthetic construct
  source 1..30: mol_type = other DNA
aataaatcataatgtagctgaggatgcctg

SEQ ID NO: 59 - DNA, 29 bp, synthetic construct
  source 1..29: mol_type = other DNA
aataaatcataatgtagctgaggatgcct

SEQ ID NO: 60 - DNA, 20 bp, synthetic construct
  source 1..20: mol_type = other DNA
gcttggtctgtgggactgtt

SEQ ID NO: 61 - DNA, 20 bp, synthetic construct
  source 1..20: mol_type = other DNA
gggaaatgagagggccagaa

SEQ ID NO: 62 - DNA, 20 bp, synthetic construct
  source 1..20: mol_type = other DNA
acattcttgtttggtgctcc

SEQ ID NO: 63 - DNA, 20 bp, synthetic construct
  source 1..20: mol_type = other DNA
gacctctccgtgccgtttga

SEQ ID NO: 64 - DNA, 20 bp, synthetic construct
  source 1..20: mol_type = other DNA
gctccagctcgtccggagat

SEQ ID NO: 65 - DNA, 20 bp, synthetic construct
  source 1..20: mol_type = other DNA
tgtttgcccacgacctgctg

SEQ ID NO: 66 - DNA, 20 bp, synthetic construct
  source 1..20: mol_type = other DNA
acgactccgtgtttgcccac

SEQ ID NO: 67 - DNA, 20 bp, synthetic construct
  source 1..20: mol_type = other DNA
gttcctattctctagaaagt

SEQ ID NO: 68 - DNA, 20 bp, synthetic construct
  source 1..20: mol_type = other DNA
cgtataatgtatgctatacg

SEQ ID NO: 69 - DNA, 20 bp, synthetic construct
  source 1..20: mol_type = other DNA
cctgcagcctgttgacaatt

SEQ ID NO: 70 - DNA, 20 bp, synthetic construct
  source 1..20: mol_type = other DNA
catcagagcagccgattgtc

SEQ ID NO: 71 - RNA, 19 nt, synthetic construct
  source 1..19: mol_type = other RNA
gaaggcatcctcagctaca

SEQ ID NO: 72 - DNA, 18 bp, Homo sapiens
  source 1..18: mol_type = genomic DNA
cagctacggggtgggctt

SEQ ID NO: 73 - DNA, 18 bp, synthetic construct
  source 1..18: mol_type = other DNA
cagctacagggtgggctt

(19) United States
(12) Patent Application Publication
(10) Pub. No.: US 2026/0002162 A1
(43) Pub. Date: Jan. 1, 2026

(54) THERAPEUTIC COMPOUNDS AND COMPOSITIONS

(71) Applicant: SISAF LTD, Guildford (GB)

(72) Inventors: Roghieh Suzanne SAFFIE-SIEBERT, Guildford (GB); Anna-Maria TETI, Riano, Rome (IT); Flavia Maria SUTERA, Guildford (GB); Antonio MAURIZI, L'Aquila (IT)

(73) Assignee: SISAF LTD, Guildford (GB)

(21) Appl. No.: 18/869,805

(22) PCT Filed: May 31, 2023
(86) PCT No.: PCT/GB2023/051440

§ 371 Date: Nov. 26, 2024

(30) Foreign Application Priority Data
May 31, 2022 (GB) 2208022.0

(51) Int. Cl. C12N 15/113; A61K 31/713; A61K 47/02; A61P 19/04

(52) U.S. Cl. CPC C12N 15/1138; A61K 31/713; A61K 47/02; A61P 19/04; C12N 2310/14; C12N 2310/533

(57) ABSTRACT

The present invention concerns siRNA molecules which are specific for mRNA encoding a mutant form of the PGFR3 (Fibroblast Growth Factor Receptor 3) protein. More particularly, this invention concerns such siRNA molecules for use in the treatment of achondroplasia.

### SEQUENCE LISTING

The instant application contains a Sequence Listing which has been submitted electronically in XML format and is hereby incorporated by reference in its entirety. Said XML copy, created on Sep. 24, 2025, is named 6223-24_SL.xml and is 101,901 bytes in size.

### FIELD OF THE INVENTION

The present invention concerns small interfering (si) RNA molecules which are specific for mRNA encoding a mutant form of the FGFR3 (Fibroblast Growth Factor Receptor 3) protein. More particularly, this invention concerns such siRNA molecules for use in the treatment of achondroplasia.

### BACKGROUND OF THE INVENTION

Achondroplasia (ACH) is a type of skeletal dysplasia caused by the FGFR3 (Fibroblast Growth Factor Receptor 3) gene, and is the most common cause of disproportionate short stature in humans. FGFR3 mutations are also implicated in other skeletal dysplasias, such as hypochondroplasia and thanatophoric dysplasia. All cases of ACH arise from autosomal dominant mutations, with the missense mutation p.Gly380Arg (localised in the FGFR3 transmembrane domain) accounting for around 97% of cases. (Shiang R et al. (1994) Cell 78:335-342, DOI: 10.1016/0092-8674 (94) 90302-6; Ornitz D M and Legeai-Mallet L (2017) Developmental Dynamics 246:291-309, DOI: 10.1002/DVDY.24479).

ACH is a rare disease, estimated to affect about 250,000 people worldwide. If left untreated, it is accompanied by multiple orthopaedic and neurological complications; achondroplasia patients often also suffer from chronic pain, which can have a profound negative impact on quality of life (Pauli R M (2019) Orphanet Journal of Rare Diseases 14:1, https://doi.org/10.1186/s13023-018-0972-6). There are currently few therapies available for achondroplasia, and to be effective, these must be administered within a limited time-frame from birth to puberty. (Ornitz D M and Legeai-Mallet L (2017) Developmental Dynamics 246:291-309, DOI: 10.1002/DVDY.24479).

Clinically used treatments for achondroplasia are limited to invasive orthopaedic surgeries, such as hip or knee joint replacement and limb lengthening procedures, and a small number of pharmaceutical interventions including growth hormone therapy. Limb lengthening surgery involves cutting cortical long bones (femur and/or tibia), followed by gradual stretching of the limb over many months to increase bone length. While the procedure is effective at increasing body height, it is time-consuming, painful, and associated with complications including infection, muscle problems, and increased fracture risk. Growth Hormone (GH) therapy involves the administration of recombinant human growth hormone (rhGH). This has been shown to result in modest growth gains, which increase further if rhGH is co-administered with thyroid hormone. However, GH therapy involves daily subcutaneous injections and is thought to be ineffective in cases with spinal and lower limb deformations. Importantly, these treatments are not curative and may have further, negative impacts on patients' quality of life if complications arise. (Wrobel W et al. (2021) Int. J. Mol. Sci. 22:5573, DOI: https://doi.org/10.3390/ijms22115573).

Some alternative, non-surgical therapies are in development, but most are still in trials or at the pre-clinical stage. The most advanced of these is BioMarin's vosoritide, an analogue of CNP (C-type Natriuretic Peptide, which is involved in normal longitudinal growth). Vosoritide was approved for treating achondroplasia in 2021, by both the FDA and EMA. Others include small molecules targeting FGFR3 signalling, such as the tyrosine kinase inhibitor infigratinib (NVP-BGJ398/BGJ398) which is currently in phase II clinical trials (NCT04265651). However, these therapies are not targeted therapies and they reduce overall FGFR3 signalling. In addition, they may have varying effectiveness against the different symptoms associated with achondroplasia. Although these treatments are effective at increasing bone length, their effects on other important aspects, such as disproportionality, axial skeleton and foramen magnum narrowing (all of which can be associated with further complications) are still unconfirmed. In general, since achondroplasia therapy is long-term, there is also a need for treatments deliverable in a form suitable for paediatric patients, with side effects minimized to the level of dose tolerance.

There is therefore still a need for novel therapies for achondroplasia and other skeletal dysplasias that are reliable and effective, but do not impede patients' quality of life. The present invention addresses the aforementioned need.

### SUMMARY OF THE INVENTION

The present invention provides, according to a first aspect, an siRNA molecule which comprises at least 17 consecutive nucleotides selected from the sequence

(SEQ ID NO: 1)
GCAGGCAUCCUCAGCUACX_{M}GGGUGGGCUUCUUCCUGU,

wherein X_{M} is selected from the nucleotides A and C, wherein said consecutive nucleotides selected from SEQ ID NO: 1 must include the X_{M} nucleotide which is found at position 19 of SEQ ID NO: 1; or a variant of said siRNA molecule having at least 1 and no more than 6 nucleotide substitutions with respect to SEQ ID NO: 1, provided that the X_{M} nucleotide at the position of said variant siRNA molecule which corresponds to position 19 of SEQ ID NO: 1 is not substituted and is thus selected from the nucleotides A and C, and wherein said siRNA molecule is not an siRNA of length 19 nucleotides having the sequence GAAGGCAUCCUCAGCUACA (SEQ ID NO: 71).

According to a second aspect of the invention, there is provided an siRNA molecule which comprises at least 17 consecutive nucleotides selected from the sequence GX₁AGX₂X₃AUCCX₄CX₅X₆X₇UX₈X₉X_{M}X₁₀X₁₁X₁₂X₁₃X₁₄GX₁₅CX₁₆UCX₁₇UX₁₈CU GU (SEQ ID NO: 14), wherein X_{M} is selected from the nucleotides A and C, wherein said consecutive nucleotides selected from SEQ ID NO: 14 must include the X_{M} nucleotide which is found at position 19 of SEQ ID NO: 14; wherein X₁ is selected from C and U, X₂ is selected from G and U, X₃ is selected from C and U, X₄ is selected from A and U, X₅ is selected from A and U, X₆ is selected from G and U, X₇ is selected from C, G and U, X₈ is selected from A and U, X₉ is selected from C and U, X₁₀ is selected from G, A and U, X₁₁ is selected from G and U, X₁₂ is selected from G and A, X₁₃ is selected from A and U, X₁₄ is selected from G and C, X₁₅ is selected from G and C, X₁₆ is selected from A and U, X₁₇ is selected from A and U and X₁₈ is selected from C and U.

According to a third aspect of the invention, there is provided a method of manufacturing an siRNA molecule or variant thereof as disclosed herein comprising the step of synthesising said siRNA molecule or variant thereof.

According to a fourth aspect of the invention, there is provided a pharmaceutical composition comprising an siRNA molecule or variant thereof as disclosed herein, and a pharmaceutically acceptable carrier.

According to a fifth aspect of the invention, there is provided a complex comprising:
(i) a lipid particle comprising hydrolysable silicon;
(ii) an siRNA or variant thereof as disclosed herein which is associated with said lipid particle.

According to a sixth aspect of the invention, there is provided a complex comprising:
(i) a particle comprising hydrolysable silicon;
(ii) optionally, one or more lipids; and
(iii) an siRNA or variant thereof as disclosed herein which is associated with said particle.

According to a seventh aspect of the invention, there is provided a complex comprising:
(i) a particle comprising hydrolysable silicon;
(ii) optionally, one or more lipids; and
(iii) an siRNA which targets an mRNA encoding a mutant FGFR3 protein, wherein said siRNA targets a portion of the mRNA sequence which includes nucleotides encoding a Gly to Arg mutation at position 380 of the protein, and wherein said siRNA is associated with said particle.

According to a eighth aspect of the invention, there is provided an siRNA molecule or variant thereof, a pharmaceutical composition, or a complex as disclosed herein, for use as a medicament.

According to an ninth aspect of the invention, there is provided an siRNA molecule or variant thereof, a pharmaceutical composition, or a complex as disclosed herein, for use in the treatment of achondroplasia.

According to a tenth aspect of the invention, there is provided an siRNA molecule or variant thereof, a pharmaceutical composition, or a complex as disclosed herein, for use in downregulating the expression of FGFR3 protein carrying a Gly to Arg mutation at position 380 (G380R).

According to a eleventh aspect of the invention, there is provided a method of treating achondroplasia in a mammal in need thereof, comprising administering to said mammal a pharmaceutically effective dose of an siRNA molecule or variant thereof, a pharmaceutical composition, or a complex as disclosed herein.

It will of course be appreciated that features described in relation to one aspect of the present invention may be incorporated into other aspects of the present invention. For example, the methods, pharmaceutical compositions and complexes of the invention may incorporate any of the features described with reference to the siRNA molecules (or variants thereof) of the invention and vice versa.

### DESCRIPTION OF THE DRAWINGS

FIG. 1 shows the generation of FGFR expression vectors.

(A) Scheme illustrating the structure of the FGFR3 GFP expressing vector.

(B) Electropherogram derived from Sanger sequencing of FGFR3^{WT} (top) and FGFR3^{G380R} expression vectors (bottom), showing the G>A transition in the mutated gene. Figure discloses SEQ ID NOS 72-73, respectively, in order of appearance.

FIG. 2 shows GFP expression at mRNA and protein level in HEK-FGFR3^{WT} and HEK-FGFR3^{G380R} cells. HEK293 cells were transfected with GFP-tagged vector carrying the wild type (HEK-FGFR3^{WT}) or the mutant (HEK-FGFR3^{G380R}) FGFR3 genes. Real time RT-PCR (A) and fluorescence microscopy (B) were used to assess GFP expression. Gene expression was normalised by human GAPDH.

FIG. 3 shows initial in vitro screening results for candidate FGFR3^{G380R}-specific siRNAs. HEK293 cells transfected with the GFP-tagged vector carrying the wild type (HEK-FGFR3^{WT}) or the mutant (HEK-FGFR3^{G380R}) FGFR3 genes were treated with 100 nM of the indicated FGFR3^{G380R}-siRNAs for 48 hours. At the end of the experiment the RNA was isolated and the GFP gene expression was evaluated by real time RT-PCR in (A) mutant and (B) wild type HEK-FGFR3 cells. Gene expression was normalised by human GAPDH. Data for each siRNA represent the arithmetic mean+/−standard deviation of 3 or 4 independent experiments. The p-value was calculated using a Multiple Comparison ANOVA (MC-ANOVA) without any correction.

FIG. 4 is a scheme illustrating the structure of the human FGFR3^{G380R} targeting vector and homologous recombination in the mouse locus, which is used to generate a knock-in (KI) mouse model of achondroplasia.

FIG. 5 shows (A) a schematic and (B) a restriction digest of the human FGFR3^{G380R} targeting vector PBS-DTA/FGFR3 (NM_000142) MUT G380R.

FIG. 6 shows results of PCR analysis of ESC clones using the primer pairs listed in Table 8.

FIG. 7A-D show the results of PCR analysis of tail biopsy samples from litters produced by crossing chimaeras to wild-type animals. NEO+HT denotes animals positive for neomycin cassette (i.e. carrying the desired G380R mutation), NEO-denotes neomycin cassette negative (i.e. not carrying the desired G380R mutation).

FIG. 8A-B show the results of PCR analysis of tail biopsy samples from litters produced by crossing chimaeras directly to FlpE animals (second mating strategy). FLP-e+ denotes animals positive for FlpE (i.e. identified as potential heterozygotes carrying the desired G380R mutation), FLP-e+ denotes FlpE negative (i.e. not carrying the desired G380R mutation).

FIG. 9 shows the results of PCR tests using the primers listed in Table 11, to detect the G380R mutation.

FIG. 10 shows the results of PCR analysis of tail biopsy samples from FLP-e+ animals (samples 16 and 22) obtained from the second mating strategy, as well as representative NEO+ (sample 4) and NEO− (sample 3), to detect the G380R mutation.

FIG. 11 shows the results of PCR analysis of tail biopsy samples from additional FLP-e+ animals obtained from the second mating strategy, as well as representative NEO+ (sample 4) and NEO− (sample 3), to detect the G380R mutation.

FIG. 12 shows the results of PCR analysis of FLP-e+/G380R+ animals for the presence or absence of the neomycin cassette.

### DETAILED DESCRIPTION

ACH is caused by an autosomal dominant mutation in the FGFR3 gene, which results in the expression of a mutant gain of function FGFR3 protein comprising a Gly to Arg substitution at position 380. Patients suffering from ACH carry a mutated FGFR3 allele and a normal (non-mutated) FGFR3 allele, and thus the present inventors hypothesised that ACH could be treated using an RNAi-based approach. In particular, the present inventors developed a mutation-driven targeted therapy based on the use of siRNAs, which are able to complement the mutant FGFR3 mRNA leading its degradation.

Thus, the present invention is in part directed to siRNA molecules which are able to target and downregulate mRNAs encoding the mutant FGFR3 protein. Such siRNA molecules are thus useful for treating or preventing ACH. Advantageously, the siRNA molecules of the present invention are able to selectively target and downregulate messenger RNAs (mRNAs) encoding the mutant FGFR3 protein, i.e. the siRNA molecules downregulate the mutant mRNA, whilst not substantially downregulating the expression of the wild-type mRNA (encoded by the normal, non-mutated FGFR3 allele). Advantageously, the siRNA molecules of the present invention are able to differentially impact the amounts of the mutated and wild-type/normal mRNAs present in a cell such that the amount of the wild-type/normal mRNA in the cell is greater than the amount of the mutated mRNA in the cell. This may be achieved by downregulating the amount of the mutated mRNA in the cell, whilst not substantially downregulating the amount of the wild-type mRNA in the cell, or it may be achieved by upregulating the amount of wild-type mRNA in the cell, whilst not substantially downregulating the amount of the mutant mRNA in the cell, or it may be achieved by both downregulating the amount of the mutated mRNA in the cell, whilst also upregulating the amount of wild-type mRNA in the cell.

The present inventors accordingly designed siRNAs which specifically target the mutant mRNA. This was achieved by designing siRNAs which are complementary to the region of the mutant mRNA which comprises the point mutation encoded by the mutated human FGFR3 gene. In certain embodiments, the siRNA design strategy of the present inventors includes the addition of a further nucleotide mismatch compared to the corresponding target sequence of the mutant FGFR3 mRNA in order to increase siRNA specificity. Such siRNA molecules would accordingly have two nucleotide mismatches with respect to the wild-type FGFR3 mRNA sequence (the point mutation which causes disease and an additional nucleotide mismatch).

The present inventors demonstrate herein that they were able to design siRNAs sequences that can bind and degrade only the mutant FGFR3 mRNA without substantially affecting the normal/wild-type FGFR3 mRNA. The latter can thus be translated into normal protein which is sufficient to restore normal FGFR3 function. This is known as “haplosufficiency”.

The siRNA-based strategies described herein are advantageous because they are highly specific for mutant FGFR3, with no or minimal off-target effects when compared with other therapeutic options including blocking antibodies, aptamers, decoy receptors and inhibitors of the tyrosine kinase activity. In addition, the siRNA molecules are more specific than other RNAi molecules including miRNAs. Indeed, miRNAs are not specific for a single mRNA but are able to target different unrelated mRNAs simultaneously and they cannot be designed in a mutation-specific manner.

Another advantage of the approach of the present inventors is represented by a low or absent risk of overdose. Indeed, since the present inventors advantageously target mutant FGFR3 signalling with minimal or absent downregulation of normal FGFR3, the maximum effect of the siRNAs should be to normalise FGFR3 signalling.

As described above, siRNAs cause gene silencing by targeting mRNA. siRNAs are short (usually around 19-25 nucleotide) double-stranded RNA molecules that work by co-opting the RNA interference response. This distinguishes them from single-stranded antisense oligonucleotides (ASOs), another tool used for oligonucleotide-induced gene silencing, which bind directly to mRNA. When an siRNA enters the cell, it is first recognised and processed by the protein machinery of the RNA induced silencing complex (RISC). The siRNA strand complementary to the target mRNA (also known as the ‘guide strand’) is then incorporated into the RISC. Once the guide strand binds to a target mRNA, one of the proteins in RISC, Argonaute, cleaves the mRNA, which in turn triggers degradation of the cleaved mRNA by exonucleases. Expression of the target mRNA and thus the protein encoded by that mRNA are therefore both down-regulated by the siRNA.

### Definitions

The term “coding sequence (CDS)”, as used herein, refers to the portion of a gene's DNA that encodes mRNA, and specifically the strand of the DNA duplex containing the sequence equivalent to that of the corresponding mRNA (with the DNA coding sequence containing thymine in the place of uracil as found in the mRNA). This is also known as the “coding strand” or the “sense strand” of the DNA.

The term “template strand”, as used herein, refers to the strand of the DNA duplex within a gene that serves as the template for mRNA transcription, i.e. it is the strand complementary to the coding sequence or coding strand. The template strand may also be referred to as the “antisense strand”.

The term “siRNA”, as used herein, refers to a short (usually around 19-25 nucleotide) RNA molecule which is at least partially double-stranded, or may be fully double stranded, and which may be used for post-transcriptional gene silencing. An siRNA contains a first strand with a sequence identical or substantially similar to that of the target mRNA, hybridised to a second, complementary strand. The siRNA molecule need not be double-stranded along its full length, for example an advantageous siRNA molecule of the present invention may be double stranded along the length of the sequence that is complementary to the target mRNA (with allowance for the possibility of one or two mismatches with the target mRNA, as described herein), but with single-stranded overhangs at one or both 3′ ends of the double-stranded molecule (wherein said single-stranded overhangs may or may not be complementary to the target mRNA). The sequences of the siRNA molecules disclosed herein are notated with respect to the first strand, i.e. the sequence identical or substantially similar to that of the target mRNA. In certain embodiments, an siRNA molecule as described herein may have an overhang of single-stranded sequence at one or both ends of the molecule. For example, an siRNA molecule as described herein may have an overhang at one or both 3′ends of the double-stranded section of the siRNA molecule. Said overhang(s) may advantageously be one or two nucleotides long, with said nucleotides being an extension of the sequence which is complementary for the target mRNA or with said nucleotides being non-complementary sequence, for example UU overhang or a deoxythymidine dinucleotide (dTdT) overhang. If overhangs are present at both 3′ ends, said overhangs may be symmetric or they may be asymmetric.

References to GFP herein should be understood as references to Enhanced Green Fluorescent Protein (EGFP).

siRNA Molecules

In an aspect, the present invention provides an siRNA molecule which comprises at least 17 consecutive nucleotides selected from the sequence

(SEQ ID NO: 1)
GCAGGCAUCCUCAGCUACX_{M}GGGUGGGCUUCUUCCUGU,

wherein X_{M} is selected from the nucleotides A and C, wherein said consecutive nucleotides selected from SEQ ID NO: 1 must include the X_{M} nucleotide which is found at position 19 of SEQ ID NO: 1; or a variant of said siRNA molecule having at least 1 and no more than 6 nucleotide substitutions with respect to SEQ ID NO: 1, provided that the X_{M} nucleotide at the position of said variant siRNA molecule which corresponds to position 19 of SEQ ID NO: 1 is not substituted and is thus selected from the nucleotides A and C.

In an embodiment of this aspect, the present invention provides an siRNA molecule which comprises at least 17 consecutive nucleotides selected from the sequence GCAGGCAUCCUCAGCUACX_{M}GGGUGGGCUUCUUCCUGU (SEQ ID NO: 1), wherein X_{M} is selected from the nucleotides A and C, wherein said consecutive nucleotides selected from SEQ ID NO: 1 must include the X_{M} nucleotide which is found at position 19 of SEQ ID NO: 1; or a variant of said siRNA molecule having at least 1 and no more than 6 nucleotide substitutions with respect to SEQ ID NO: 1, provided that the X_{M} nucleotide at the position of said variant siRNA molecule which corresponds to position 19 of SEQ ID NO: 1 is not substituted and is thus selected from the nucleotides A and C, and wherein said siRNA molecule is not an siRNA of length 19 nucleotides having the sequence

(SEQ ID NO: 71)
GAAGGCAUCCUCAGCUACA.

In an embodiment of this aspect, there is provided an siRNA molecule which comprises at least 17 consecutive nucleotides selected from the sequence

(SEQ ID NO: 1)
GCAGGCAUCCUCAGCUACX_{M}GGGUGGGCUUCUUCCUGU,

wherein X_{M} is selected from the nucleotides A and C, wherein said consecutive nucleotides selected from SEQ ID NO: 1 must include the X_{M} nucleotide which is found at position 19 of SEQ ID NO: 1; or a variant of said siRNA molecule having at least 1 and no more than 6 nucleotide substitutions with respect to SEQ ID NO: 1, provided that the X_{M} nucleotide at the position of said variant siRNA molecule which corresponds to position 19 of SEQ ID NO: 1 is not substituted and is thus selected from the nucleotides A and C, and wherein said siRNA molecule does not consist of the sequence GAAGGCAUCCUCAGCUACA (SEQ ID NO: 71).

In an embodiment of this aspect, there is provided an siRNA molecule which comprises at least 17 consecutive nucleotides selected from the sequence

(SEQ ID NO: 1)
GCAGGCAUCCUCAGCUACX_{M}GGGUGGGCUUCUUCCUGU,

wherein X_{M} is selected from the nucleotides A and C, wherein said consecutive nucleotides selected from SEQ ID NO: 1 must include the X_{M} nucleotide which is found at position 19 of SEQ ID NO: 1; or a variant of said siRNA molecule having at least 1 and no more than 6 nucleotide substitutions with respect to SEQ ID NO: 1, provided that the X_{M} nucleotide at the position of said variant siRNA molecule which corresponds to position 19 of SEQ ID NO: 1 is not substituted and is thus selected from the nucleotides A and C, and wherein said siRNA molecule does not comprise the sequence GAAGGCAUCCUCAGCUACA (SEQ ID NO: 71).

In an embodiment of this aspect, said siRNA molecule or variant thereof comprises at least 18 or at least 19 consecutive nucleotides selected from the sequence of SEQ ID NO: 1. In another embodiment of this aspect, said siRNA molecule or variant thereof comprises at least 20, at least 21, at least 22, at least 23, at least 24, at least 25, at least 26, at least 27, at least 28, at least 29, or at least 30 consecutive nucleotides selected from the sequence of SEQ ID NO: 1.

In an embodiment of this aspect, the maximum length of the siRNA molecule or variant thereof is 30, 29, 28, 27, 26 or fewer nucleotides, for example 25 or fewer nucleotides.

In an embodiment of this aspect, the length of the siRNA molecule or variant thereof is from 17 to 25 nucleotides, preferably from 19 to 23 nucleotides. In another embodiment of this aspect, the length of the siRNA molecule or variant thereof is from 20 to 23 nucleotides. In another embodiment of this aspect, the length of the siRNA molecule or variant thereof is from 19 to 22 nucleotides, from 19 to 21 nucleotides or from 19 to 20 nucleotides. In another embodiment of this aspect, the length of the siRNA molecule is 19 nucleotides.

In an embodiment of this aspect, said variant of said siRNA molecule has at least 1 and no more than 5 nucleotide substitutions with respect to SEQ ID NO. 1, provided that the X_{M} nucleotide at the position of said variant siRNA molecule which corresponds to position 19 of SEQ ID NO: 1 is not substituted and is thus selected from the nucleotides A and C. In another embodiment of this aspect, said variant of said siRNA molecule has at least 1 and no more than 4 nucleotide substitutions with respect to SEQ ID NO. 1, provided that the X_{M} nucleotide at the position of said variant siRNA molecule which corresponds to position 19 of SEQ ID NO: 1 is not substituted and is thus selected from the nucleotides A and C. In another embodiment of this aspect, said variant of said siRNA molecule has at least 1 and no more than 3 nucleotide substitutions with respect to SEQ ID NO. 1, provided that the X_{M} nucleotide at the position of said variant siRNA molecule which corresponds to position 19 of SEQ ID NO: 1 is not substituted and is thus selected from the nucleotides A and C. In another embodiment of this aspect, said variant of said siRNA molecule has at least 1 and no more than 2 nucleotide substitutions with respect to SEQ ID NO. 1, provided that the X_{M} nucleotide at the position of said variant siRNA molecule which corresponds to position 19 of SEQ ID NO: 1 is not substituted and is thus selected from the nucleotides A and C. In another embodiment of this aspect, said variant of said siRNA molecule has only 1 nucleotide substitution with respect to SEQ ID NO. 1, provided that the X_{M} nucleotide at the position of said variant siRNA molecule which corresponds to position 19 of SEQ ID NO: 1 is not substituted and is thus selected from the nucleotides A and C.

In an embodiment of this aspect, said siRNA molecule or variant thereof is at least partially double-stranded, i.e. comprises a second strand which is substantially or completely complementary to the siRNA sequences disclosed herein. In another embodiment of this aspect, said siRNA molecule or variant thereof is double-stranded along its full length, i.e. comprises a second strand which is completely complementary to the siRNA sequences disclosed herein. siRNA molecules or variants thereof of the present invention may have an overhang of single-stranded sequence at one or both 3′ ends of the double-stranded section of the molecule. For example, an siRNA or variant thereof of the present invention may have a single-stranded overhang at one or both 3′ ends of 1, 2, 3 or 4 nucleotides, for instance two nucleotides. Advantageously, an siRNA molecule or variant thereof of the present invention has single-stranded overhangs at both 3′ ends of the double-stranded molecule, for instance of two nucleotides. Advantageously, the overhanging nucleotides do not comprise a G nucleotide. In another embodiment of this aspect, the overhanging nucleotides of said siRNA molecule or variant thereof are an extension of the sequence which is complementary for the target mRNA, or the overhanging nucleotides are non-complementary sequence, for example UU or deoxythymidine dinucleotide (dTdT).

In an embodiment of this aspect, X_{M} within said siRNA molecule or variant thereof is the nucleotide A. In another embodiment of this aspect, X_{M} within said siRNA molecule or variant thereof is the nucleotide C.

In an embodiment of this aspect, the length of said siRNA molecule or variant thereof is at least 19 nucleotides, and the A of the siRNA molecule or variant thereof which corresponds to position 19 of SEQ ID NO: 1 may be found at any of the positions of the siRNA molecule or variant thereof. In another embodiment of this aspect, the length of said siRNA molecule or variant thereof is 19 to 25 nucleotides, and the A of the siRNA molecule or variant thereof which corresponds to position 19 of SEQ ID NO: 1 may be found at position 1, position 6, position 9, position 15 or position 19 of the siRNA molecule or variant thereof. In another embodiment of this aspect, the length of said siRNA molecule or variant thereof is 19 to 25 nucleotides, and the A of the siRNA molecule or variant thereof which corresponds to position 19 of SEQ ID NO: 1 may be found at position 1, position 6 or position 9 of the siRNA molecule or variant thereof. In another embodiment of this aspect, the length of said siRNA molecule or variant thereof is 19 to 25 nucleotides, and the A of the siRNA molecule or variant thereof which corresponds to position 19 of SEQ ID NO: 1 may be found at position 6 of the siRNA molecule or variant thereof. In an embodiment of this aspect, said siRNA molecule or variant thereof comprises the sequence X₁X₂UAX₃X_{M}GGGUGGX₄CX₅UCUU (SEQ ID NO: 2), wherein X_{M} is selected from the group consisting of A and C and where X₁, X₂, X₃, X₄ and X₅ are each independently selected from the group consisting of A, G, C and U. In another embodiment of this aspect, said siRNA molecule or variant thereof comprises SEQ ID NO: 2, wherein X₁ is selected from G and U, X₂ is selected from C and U, X₃ is selected from C and U, X₄ is selected from G and C and X₅ is selected from A and U. In another embodiment of this aspect, said siRNA molecule or variant thereof comprises SEQ ID NO: 2 and X₄ is G. In another embodiment of this aspect, said siRNA molecule or variant thereof comprises SEQ ID NO: 2, wherein X₁ is G, X₂ is C, X₃ is selected from C and U, X₄ is G and X₅ is selected from A and U. In another embodiment of this aspect, said siRNA molecule is selected from the group consisting of GCUAUAGGGUGGGCUUCUU (SEQ ID NO: 3) and GCUACAGGGUGGGCAUCUU (SEQ ID NO: 4). In an embodiment of this aspect, said siRNA molecule or variant thereof comprises the sequence X_{M}X₁X₂GUGGGCUUCX₃UX₄CUGU (SEQ ID NO: 5), wherein X_{M} is selected from the group consisting of A and C and wherein X₁, X₂, X₃ and X₄ are each independently selected from the group consisting of A, G, C and U. In another embodiment of this aspect, said siRNA molecule or variant thereof comprises SEQ ID NO: 5, wherein X₁ is selected from G and A, X₂ is selected from G and U, X₃ is selected from A and U and X₄ is selected from C and U. In another embodiment of this aspect, said siRNA molecule or variant thereof comprises SEQ ID NO: 5 and X₃ is U. In another embodiment of this aspect, said siRNA molecule or variant thereof comprises SEQ ID NO: 5, wherein X₁ is selected from A and G, X₂ is G, X₃ is U and X₄ is selected from C and U. In another embodiment of this aspect, said siRNA molecule is selected from the group consisting of AAGGUGGGCUUCUUCCUGU (SEQ ID NO: 6) and AGGGUGGGCUUCUUUCUGU (SEQ ID NO: 7)

In an embodiment of this aspect, said siRNA molecule or variant thereof comprises the sequence X₁CAGCUAX₂X_{M}X₃GGX₄X₅GGCUU (SEQ ID NO: 8), wherein X_{M} is selected from the group consisting of A and C and wherein X₁, X₂, X₃, X₄ and X₅ are each independently selected from the group consisting of A, G, C and U. In another embodiment of this aspect, said siRNA molecule or variant thereof comprises SEQ ID NO: 8, wherein X₁ is selected from A and U, X₂ is selected from C and U, X₃ is selected from G and U, X₄ is selected from A and U and X₅ is selected from C and G. In another embodiment of this aspect, said siRNA molecule or variant thereof comprises SEQ ID NO: 8 and X₂ is C. In another embodiment of this aspect, said siRNA molecule is selected from the group consisting of

(SEQ ID NO: 9)
UCAGCUACAGGGUCGGCUU
and
(SEQ ID NO: 10)
UCAGCUACAGGGAGGGCUU

In an embodiment of this aspect, said siRNA molecule or variant thereof comprises the sequence X₁CAUCCUCAGCX₂X₃X₄X_{M}X₅GX₆U (SEQ ID NO: 11), wherein X_{M} is selected from the group consisting of A and C and wherein X₁, X₂, X₃, X₄, X₅ and X₆ are each independently selected from the group consisting of A, G, C and U. In another embodiment of this aspect, said siRNA molecule or variant thereof comprises SEQ ID NO: 8, wherein X₁ is selected from G and U, X₂ is selected from C and U, X₃ is selected from A and U, X₄ is selected from C and U, X₅ is selected from G and U and X₆ is selected from G and A. In another embodiment of this aspect, said siRNA molecule has the sequence has the sequence GCAUCCUCAGCUAUAGGGU (SEQ ID NO: 12).

In an embodiment of this aspect, said siRNA molecule or variant thereof comprises the sequence GX₁AGGX₂AUCCUCX₃GX₄UAX₅X_{M} (SEQ ID NO: 13), wherein X_{M} is selected from the group consisting of A and C and wherein X₁, X₂, X₃, X₄ and X₅ are each independently selected from the group consisting of A, G, C and U. In another embodiment of this aspect, said siRNA molecule or variant thereof comprises SEQ ID NO: 13, wherein X₁ is selected from C and U, X₂ is selected from C and U, X₃ is selected from A and U, X₄ is selected from G and C and X₅ is selected from C and U.

In an embodiment of this aspect, said siRNA molecule or variant thereof comprises a sequence falling within the definition of SEQ ID NO: 2, SEQ ID NO: 5, SEQ ID NO: 8, SEQ ID NO: 11, or SEQ ID NO: 13, wherein in each of SEQ ID NO: 2, SEQ ID NO: 5, SEQ ID NO: 8, SEQ ID NO: 11 and SEQ ID NO: 13, X_{M} is A. In this context, a reference to a sequence falling within the definition of SEQ ID NO: 2, SEQ ID NO: 5, SEQ ID NO: 8, SEQ ID NO: 11, or SEQ ID NO: 13 refers to a sequence having the full length of SEQ ID NO: 2, SEQ ID NO: 5, SEQ ID NO: 8, SEQ ID NO: 11, or SEQ ID NO: 13, i.e. 19 nucleotides.

In an embodiment of this aspect, said siRNA molecule comprises a sequence selected from the group consisting of:

| | Sequence |
|---|---|
| | (Bold nucleotides indicate G1138A |
| | mutation; Underlined nucleotides |
| | indicate additional mismatches |
| | relative to the human wild-type |
| SEQ ID NO. | FGFR3 sequence) |
| 15 | GCAGGCAUCCUCAGCUACA |
| 16 | GCAGGCAUCCUCAGCUAUA |
| 17 | GCAGGCAUCCUCAGGUACA |
| 18 | GCAGGCAUCCUCUGCUACA |
| 19 | GCUACAGGGUGGGCUUCUU |
| 3 | GCUAUAGGGUGGGCUUCUU |
| 20 | GCUACAGGGUGGCCUUCUU |
| 4 | GCUACAGGGUGGGCAUCUU |
| 21 | AGGGUGGGCUUCUUCCUGU |
| 6 | AAGGUGGGCUUCUUCCUGU |
| 7 | AGGGUGGGCUUCUUUCUGU |
| 22 | AGGGUGGGCUUCAUCCUGU |
| 23 | UCAGCUACAGGGUGGGCUU |
| 24 | UCAGCUAUAGGGUGGGCUU |
| 9 | UCAGCUACAGGGUCGGCUU |
| 10 | UCAGCUACAGGGAGGGCUU |
| 25 | GCAUCCUCAGCUACAGGGU |
| 12 | GCAUCCUCAGCUAUAGGGU |
| 26 | GCAUCCUCAGCUUCAGGGU |
| 27 | GCAUCCUCAGCCACAGGGU |
| 28 | GUAGGCAUCCUCAGCUACA |
| 29 | GCAGGUAUCCUCAGCUACA |
| 30 | GUUACAGGGUGGGCUUCUU |
| 31 | UCUACAGGGUGGGCUUCUU |
| 32 | AGUGUGGGCUUCUUCCUGU |
| 33 | UCAUCCUCAGCUACAGGGU |
| 34 | GCAUCCUCAGCUACAUGGU |
| 35 | GCAUCCUCAGCUACAGGAU |
| 36 | ACAGCUACAGGGUGGGCUU |
| 37 | UCAGCUACAUGGUGGGCUU |

In another embodiment of this aspect, the present invention includes an siRNA molecule selected from the above table in which the A nucleotide indicated in bold has been replaced by a C nucleotide.

Further siRNA molecules can be designed or the siRNA molecules described herein can be modified based on one or more of the following principles: (i) aim for a GC content in the range 30-50%; (ii) avoid target sequences with more than 16-17 contiguous base pairs of homology to other coding sequences, as identified by BLAST (http://blast.nebi.nlm.nih.gov/Blast.cgi) and (iii) include a UU dinucleotide overhang at the 3′ end.

According to a second aspect of the invention, there is provided an siRNA molecule which comprises at least 17 consecutive nucleotides selected from the sequence GX₁AGX₂X₃AUCCX₄CX₅X₆X₇UX: X₉X_{M}X₁₀X₁₁X₁₂X₁₃X₁₄GX₁₅CX₁₆UCX₁₇UX₁₈CU GU (SEQ ID NO: 14), wherein X_{M} is selected from the nucleotides A and C, wherein said consecutive nucleotides selected from SEQ ID NO: 14 must include the X_{M} nucleotide which is found at position 19 of SEQ ID NO: 14; wherein X₁ is selected from C and U, X₂ is selected from G and U, X₃ is selected from C and U, X₄ is selected from A and U, X₅ is selected from A and U, X₆ is selected from G and U, X₇ is selected from C, G and U, X₈ is selected from A and U, X₉ is selected from C and U, X₁₀ is selected from G, A and U, X₁₁ is selected from G and U, X₁₂ is selected from G and A, X₁₃ is selected from A and U, X₁₄ is selected from G and C, X₁₅ is selected from G and C, X₁₆ is selected from A and U, X₁₇ is selected from A and U and X₁₈ is selected from C and U.

In an embodiment of this aspect, said siRNA molecule or variant thereof comprises at least 18 or at least 19 consecutive nucleotides selected from the sequence of SEQ ID NO: 14. In another embodiment of this aspect, said siRNA molecule or variant thereof comprises at least 20, at least 21, at least 22, at least 23, at least 24, at least 25, at least 26, at least 27, at least 28, at least 29, or at least 30 consecutive nucleotides selected from the sequence of SEQ ID NO: 14.

In an embodiment of this aspect, the maximum length of the siRNA molecule or variant thereof is 30 or fewer, 29 or fewer, 28 or fewer, 27 or fewer, 26 or fewer or 25 or fewer nucleotides. In another embodiment of this aspect, the length of the siRNA molecule or variant thereof is from 17 to 25 nucleotides, from 18 to 24 nucleotides or from 19 to 23 nucleotides. In another embodiment of this aspect, the length of the siRNA molecule or variant thereof is from 19 to 22 nucleotides, from 19 to 21 nucleotides or from 19 to 20 nucleotides. In another embodiment of this aspect, the length of the siRNA molecule is 19 nucleotides.

In an embodiment of this aspect, said siRNA molecule is at least partially double-stranded. In another embodiment of this aspect, said siRNA molecule or variant thereof is double-stranded along its full length. In another embodiment of this aspect, said siRNA molecule or variant thereof has an overhang of single-stranded sequence at one or both ends of the molecule. In another embodiment of this aspect, said siRNA or variant thereof has a single-stranded overhang at the 3′ end of 1, 2, 3 or 4 nucleotides. Advantageously, the overhanging nucleotides does not comprise a G nucleotide. In another embodiment of this aspect, said siRNA molecule or variant thereof comprises a 3′ overhang with the sequence UU.

In an embodiment of this aspect, X_{M} within said siRNA molecule or variant thereof is the nucleotide A. In another embodiment of this aspect, X_{M} within said siRNA molecule or variant thereof is the nucleotide C.

In an embodiment of this aspect, X₁₅ within said siRNA molecule or variant thereof is the nucleotide G and X₁₇ is the nucleotide U.

In an embodiment of this aspect, said siRNA molecule comprises a sequence selected from the group consisting of:

| | Sequence |
|---|---|
| | (Bold nucleotides indicate G1138A |
| | mutation; Underlined nucleotides |
| | indicate additional mismatches |
| | relative to the human wild-type |
| SEQ ID NO. | FGFR3 sequence) |
| 15 | GCAGGCAUCCUCAGCUACA |
| 16 | GCAGGCAUCCUCAGCUAUA |
| 17 | GCAGGCAUCCUCAGGUACA |
| 18 | GCAGGCAUCCUCUGCUACA |
| 19 | GCUACAGGGUGGGCUUCUU |
| 3 | GCUAUAGGGUGGGCUUCUU |
| 20 | GCUACAGGGUGGCCUUCUU |
| 4 | GCUACAGGGUGGGCAUCUU |
| 21 | AGGGUGGGCUUCUUCCUGU |
| 6 | AAGGUGGGCUUCUUCCUGU |
| 7 | AGGGUGGGCUUCUUUCUGU |
| 22 | AGGGUGGGCUUCAUCCUGU |
| 23 | UCAGCUACAGGGUGGGCUU |
| 24 | UCAGCUAUAGGGUGGGCUU |
| 9 | UCAGCUACAGGGUCGGCUU |
| 10 | UCAGCUACAGGGAGGGCUU |
| 25 | GCAUCCUCAGCUACAGGGU |
| 12 | GCAUCCUCAGCUAUAGGGU |
| 26 | GCAUCCUCAGCUUCAGGGU |
| 27 | GCAUCCUCAGCCACAGGGU |
| 28 | GUAGGCAUCCUCAGCUACA |
| 29 | GCAGGUAUCCUCAGCUACA |
| 30 | GUUACAGGGUGGGCUUCUU |
| 31 | UCUACAGGGUGGGCUUCUU |
| 32 | AGUGUGGGCUUCUUCCUGU |
| 33 | UCAUCCUCAGCUACAGGGU |
| 34 | GCAUCCUCAGCUACAUGGU |
| 35 | GCAUCCUCAGCUACAGGAU |
| 36 | ACAGCUACAGGGUGGGCUU |
| 37 | UCAGCUACAUGGUGGGCUU |

In another embodiment of this aspect, the present invention includes an siRNA molecule selected from the above table in which the A nucleotide indicated in bold has been replaced by a C nucleotide.

Methods of siRNA Synthesis

According to a third aspect of the invention, there is provided a method of manufacturing an siRNA molecule or variant thereof as disclosed herein comprising the step of synthesising said siRNA molecule or variant thereof.

Methods of siRNA synthesis are known in the art and the skilled person will be able to select an appropriate method of synthesising the siRNA molecules or variants thereof disclosed herein. Synthesis methods may involve in vitro approaches including: chemical synthesis (e.g. via the GeneAssist™ Custom siRNA Builder commercial service provided by ThermoFisher) or in vitro transcription of a suitable DNA template (e.g. using the Ambion™ Silencer™ siRNA Construction Kit provided by ThermoFisher). Alternatively, synthesis of siRNAs can be achieved via the introduction of DNA-based expression systems into cells, e.g. siRNA expression plasmids such as the pSilencer™ siRNA expression vector from Thermofisher, viral vectors, or PCR-generated siRNA expression cassettes such as those described in Castanotto D., (2002), RNA 8:1454-60. Such systems allow the siRNA molecules to be directly expressed in vivo without the need to transfect the siRNA molecules into the cells. Therefore, in an embodiment of this third aspect, said step of synthesising said siRNA molecule or variant thereof may be performed using chemical synthesis, in vitro transcription, or cell-based siRNA expression systems (for example siRNA expression plasmids, viral vectors, or PCR siRNA expression cassettes). In an embodiment of this aspect, said step of synthesising said siRNA molecule or variant thereof is performed using chemical synthesis or in vitro transcription. In an embodiment of this aspect, said step of synthesising said siRNA molecule or variant thereof is performed using chemical synthesis.

### Pharmaceutical Compositions

According to a fourth aspect of the invention, there is provided a pharmaceutical composition comprising an siRNA molecule or variant thereof as disclosed herein, and a pharmaceutically acceptable carrier.

Pharmaceutically acceptable carriers suitable for use according to the fourth aspect of the invention, and their formulations, are known in the art and described in standard formulation treatises, such as Remington's Pharmaceutical Sciences by E. W. Martin, or Wang, Y. J. or Hanson, M. A., Journal of Parenteral Science and Technology, Technical Report No. 10, Supp. 42: 2S, 1988. Further examples of such carriers are set out in, for example, the 2019 European Pharmacopeia (Ph. Eur.) and the Handbook of Pharmaceutical Excipients (9th edition, 2020; Pharmaceutical Press (UK) and American Pharmaceutical Association (US)). The skilled person will be able to select an appropriate carrier for a pharmaceutical composition comprising an siRNA molecule or variant thereof as disclosed herein.

In an embodiment of this aspect, said pharmaceutical composition comprises a polymer selected from the group consisting of: hydroxypropyl methylcellulose (HPMC), hydroxypropyl cellulose, methylcellulose, carbomer, hyaluronan, chitosan, N-trimethyl chitosan, N-carboxymethyl chitosan, sodium carboxymethylcellulose, polygalacturonic acid, sodium alginate, xanthan gum, xyloglucan gum, scleroglucan, polyvinyl alcohol, and polyvinyl pyrrolidine.

In an embodiment of this aspect, said pharmaceutical composition comprises a complex as described herein.

Particles Comprising Silicon Complexed with siRNA

Advantageously, the siRNA molecules or variants thereof of the present invention are delivered, e.g. in vivo, using lipid particles comprising hydrolysable silicon. Therefore, according to a fifth aspect of the invention, there is provided a complex comprising:
(i) a lipid particle comprising hydrolysable silicon;
(ii) an siRNA or variant thereof as disclosed herein which is associated with said lipid particle.

As explained below, the term “lipid particle comprising hydrolysable silicon” refers to a particle comprising hydrolysable silicon, and one or more lipids.

In an embodiment of this aspect, said lipid particle comprising hydrolysable silicon comprises hydrolysable elemental silicon.

In a sixth aspect of the invention, there is provided a complex comprising:
(i) a particle comprising hydrolysable silicon;
(ii) optionally, one or more lipids; and
(iii) an siRNA or variant thereof as disclosed herein which is associated with said particle.

In an embodiment of this aspect, said particle comprising hydrolysable silicon comprises hydrolysable elemental silicon.

In an embodiment of these fifth and sixth aspects, the siRNA or variant thereof is associated with said particle in that it is bound to said particle, for example in that it is electrostatically bound to said particle.

In an embodiment of these fifth and sixth aspects, the particle comprising hydrolysable silicon in the complex is a nanoparticle, for example a silicon stabilised hybrid lipid nanoparticle (sshLNP™) as manufactured by SiSaf Ltd (Guildford, UK). As used herein, the term “silicon stabilised hybrid lipid nanoparticle” refers to nanoparticles, comprising silicon and at least some lipid, which have at least some micellar character. Thus, the lipid molecules present tend to arrange themselves into a micelle or micelle-like structure (i.e., a structure having at least some micellar character). Micellar or micellar-like lipid particles are distinct from liposomes which comprise a lipid bilayer, but the term “micellar or micellar-like lipid particles” as used herein admits at least some liposome-type structures within the particle and, for this reason, they may be referred to as “hybrid” particles.

Silicon stabilised hybrid lipid nanoparticles are generically disclosed in GB2210794.0 (which is incorporated herein by reference and which may be obtained on publication or from the publicly available file on publication of other patent applications, for example international patent applications claiming priority from it). Advantageous methods of manufacturing silicon stabilised hybrid lipid nanoparticles are generically disclosed in GB2300914.5 (which is also incorporated herein by reference and which may also be obtained on publication or from the publicly available file on publication of other patent applications, for example international patent applications claiming priority from it).

Conventional lipid particles, including lipid nanoparticles (LNPs) have shown some promise as vectors for the delivery of therapeutic nucleic acid and in particular for the delivery of therapeutic RNA. RNA is especially prone to degradation during formulation and storage. Various pharmaceutical compositions that seek to limit the extent of RNA degradation have been proposed. One way of limiting RNA degradation is to seek to encapsulate the RNA in a protective micellar lipid. For example, EP3677567A1 discloses lipid particles which have mRNA molecules encapsulated within the particles.

However, various challenges are associated with the use of LNPs for RNA delivery. Among these challenges is the requirement to form micellar or micellar-like lipid particles at an elevated temperature. The precise temperature required depends on the lipids used but typically temperatures of about 60° C. need to be employed. Such elevated temperatures cause significant degradation of RNA and other fragile active ingredients. This can be countered, to an extent, by use of modified active ingredients, for example modified RNA. It is notable that U.S. Pat. No. 9,504,651 B2 discloses a method of forming micellar lipid particles “around” an mRNA wherein at least 70% of the mRNA is encapsulated. Of the unencapsulated 30%, it appears that much is degraded.

There are also related challenges in keeping micellar or micellar-like lipid particles “stable”. Small lipid particles may have a tendency to coalesce into larger particles, as discussed in GB2210794.0 and GB2300914.5. Thus, improving “stability” includes both countering the tendency of small micellar or micellar-like lipid particles to coalesce into larger particles and also countering their tendency to lose the charges of the charged lipids which are part of the micellar or micellar-like lipid particle. Much work has been carried out in this field on producing novel lipids with advantageous properties, and in developing formulations of multiple lipids which have advantageous properties in their ability to form stable micellar or micellar-like particles.

In silicon stabilised hybrid lipid nanoparticles, silicon particles, which themselves are smaller than micelles or micelle-like structures, can be “dusted” onto the surface of the micelle or micelle-like structure and/or “into” the surface of the lipid particle (i.e., partially penetrating into the lipid particle but with a portion of the silicon particles accessible on the surface) and thereby inhibit the tendency of the lipid particles to coalesce with each other, as well as coordinating with and thereby protecting any charged lipid constituents of the lipid particle and also providing the lipid particles with a superior ability to complex with RNA.

Complexes of the present invention which are based on silicon stabilised hybrid lipid nanoparticles, especially sshLNP™, may advantageously allow high volumes of tightly condensed RNA to bind electrostatically, thus protecting the RNA from hydrolysis and prolonging its survival in vivo. Furthermore, the silicon stabilised hybrid lipid nanoparticles, especially sshLNP™, may provide a positive charge and high ζ-potential for improved electrostatic binding of RNA, which may prevent premature disassociation of RNA from the complex.

Summarising, one advantage of silicon stabilised hybrid lipid nanoparticles lies in improvements in the stability of micellar or micellar-like lipid particles.

Another advantage of silicon stabilised hybrid lipid nanoparticles, especially sshLNP™, lies in the provision of methods to improve micellar or micellar-like particle stability which are less dependent on the use of specific novel lipids which may be subject to technical challenges, supply constraints and intellectual property restrictions on their use; in particular, cationic or ionisable lipids.

By using silicon stabilised hybrid lipid nanoparticles, it is possible to form sufficiently stable delivery vehicles for RNA, with a wider range of lipids including lipids which are of lower cost and more readily available then some of the specialist lipids which may need to be used in prior art methods and products in order to obtain micellar or micellar-like lipid particles having sufficient performance.

Conversely, the silicon stabilised hybrid lipid nanoparticles may also be used with advantageous prior art “high performance” specialised lipids to achieve even more superior performance.

Thus, silicon stabilised hybrid lipid nanoparticles leverage the discovery that particles of silicon (especially particles containing hydrolysable silicon) can be used to stabilise micellar or micellar-like lipid particles, to inhibit their coalescence, to assist cationic lipids and ionisable lipids in retaining their charge and to promote the ability of the micellar or micellar-like lipid particles to protect nucleic acid, not by encapsulation but by stabilising the nucleic acid on the micellar or micellar-like lipid particle surface.

Such an arrangement advantageously allows the micellar or micellar-like lipid particles to be prepared in the absence of nucleic acid and the nucleic acid may be added to the particles only after they have been fully formed and any processes involving elevated temperature have been completed, as described in GB2210794.0 and GB2300914.5. Thus, such sshLNP™ can also advantageously be prepared, stored, and delivered to a clinic, then complexed with RNA (e.g. a siRNA or variant thereof as disclosed herein) before it is administered to a patient. The delivery vehicle is able to be stored separately from the RNA, until close to the time when it is administered to a patient. Since the delivery vehicle does not contain the RNA while it is being stored, there is no need for storage at especially cold temperatures such as below 4° C. in order specifically to stabilise the RNA.

Notably, complexes based on silicon stabilised hybrid lipid nanoparticles, particularly sshLNP™, are highly versatile because the silicon stabilised hybrid lipid nanoparticles (especially, sshLNP™) provide through ionisable mesoporous silicon a very a high surface area (>700 m²/g), a pore volume of >1 cm³/g, a stable nanostructure, a tunable pore diameter (2-10 nm), two functional surfaces (exterior and interior), control of particle size and shape and a modifiable particle surface.

Examples of sshLNP™ silicon stabilised hybrid lipid nanoparticles suitable for use in a complex of the present invention include SIS0012 (with undoped silicon), SIS0012 2LBS (with doped silicon), SIS0013 (with doped silicon), and modified versions thereof (e.g. -N, -T and -Q variants, which are preparable by adding, as a further component, 0.2 mg of NAD, tyrosine or quercetin, respectively).

The composition and production of such sshLNP™ silicon stabilised hybrid lipid nanoparticles, including SIS0012, SIS0013 and SIS0012 2LBS are described elsewhere herein. The composition and production of those sshLNP™ silicon stabilised hybrid lipid nanoparticles, as well as SIS0013-N, SIS0013-T and SIS0013-Q, are also described in UK patent application no. 2300912.9, the disclosure of which is hereby incorporated by reference in its entirety (and which may also be obtained on publication or from the publicly available file on publication of other patent applications, for example international patent applications claiming priority from it).

A complex according to these fifth and sixth aspects of the invention may be prepared by combining the siRNA molecule or variant thereof with a suitable solvent such as water, a particle comprising hydrolysable silicon, and one or more lipids. The composition may optionally further comprise one or more amino acids (for example glycine or a mixture of amino acids including glycine) and optionally one or more non-reducing disaccharides such as trehalose. A suitable preparation method may include dispersing the lipid component in a solvent such as methanol; generating a thin film of lipid by evaporating the solvent, for example in a rotary evaporator; hydrating the lipid with an aqueous solution containing activated hydrolysable silicon particles, for example particles having an average particle size less than 100 nm, a non-reducing disaccharide such as trehalose and one or more amino acid such as glycine. The composition may optionally be passed though filters, for example 0.4 and 0.1 μm filters to achieve complexation and dispersal of the particles. The composition may optionally be stored at 4° C. if required to allow further complexation to take place. A carrier prepared thus may then be complexed with an aqueous solution of the siRNA or a variant thereof at ratios ranging from 1:6 to 1:16, where 1 represents the nucleic acid. Preferred ratios are 1:8-1:12, where 1:8 usually allows a small excess of biological, and 1:12 allows a small excess of the carrier.

The hydrolysable silicon of the lipid particle of the fifth aspect may be in the form of silicon particles. The silicon particles may be pure silicon or substantially pure silicon (or pure doped silicon or substantially pure doped silicon), or another hydrolysable silicon-containing material (or another hydrolysable doped silicon-containing material). If they are not pure silicon, they contain at least 50% by weight silicon, i.e. they comprise at least 50% by weight silicon atoms based on the total mass of atoms in the particles. For example, the silicon particles may contain at least 60, 70, 80, 90 or 95% silicon. The silicon particles preferably show a rate of hydrolysis, for example in PBS buffer at room temperature, of at least 10% of the rate of hydrolysis of pure silicon particles of the same dimensions. Assays for hydrolysis of silicon-containing material are widely known in the art (see, for example, WO2011/001456, incorporated by reference herein). Although the silicon particles of the invention may contain some silica, silica is not hydrolysable silicon and at least half of the silicon atoms in the particles are in the form of elemental silicon (or doped elemental silicon).

The particle comprising hydrolysable silicon of the sixth aspect of the invention may be pure silicon or substantially pure silicon (or pure doped silicon or substantially pure doped silicon), or another hydrolysable silicon-containing material (or another hydrolysable doped silicon-containing material). If the silicon is not pure silicon, it contains at least 50% by weight silicon, i.e. it comprises at least 50% by weight silicon atoms based on the total mass of atoms in the particles. For example, the silicon may contain at least 60, 70, 80, 90 or 95% silicon. The silicon preferably shows a rate of hydrolysis, for example in PBS buffer at room temperature, of at least 10% of the rate of hydrolysis of pure silicon particles of the same dimensions. Assays for hydrolysis of silicon-containing material are widely known in the art (see, for example, WO2011/001456, incorporated by reference herein). Although the silicon of the invention may contain some silica, silica is not hydrolysable silicon and at least half of the silicon atoms are in the form of elemental silicon (or doped elemental silicon).

The particles comprising hydrolysable silicon may be nanoparticles. Nanoparticles have a nominal diameter of between about 1 to about 500 nm, for example about 1 to about 250 nm, for example about 1 to about 100 nm (e.g. about 30 nm), or for example about 5 to about 400 nm, for example about 50 to about 350 nm, for example about 80 to about 310 nm, for example about 100 to about 250 nm, for example about 120 to about 240 nm, for example about 150 to about 220 nm, for example about 200 nm. They may be made of either pure silicon or a hydrolysable silicon-containing material. They are preferably porous, especially mesoporous. The nominal diameter referred to above, may refer to the mean diameter and at least 90% of total mass of particles in a sample of particles may fall within the size range specified. Particle size may be ascertained or confirmed by transmission electron microscopy (TEM), for example using the NIST-NCL Joint Assay Protocol, PCC-X, version 1.1, “Measuring the size of nanoparticles using TEM”, revised February 2010: https://tsapps.nist.gov/publication/get_pdf.cfm?pub_id=854083

Particles comprising hydrolysable silicon can be made porous by standard techniques such as contacting the particles with a hydrofluoric acid (HF)/ethanol mixture and applying a current. By varying the HF concentration and the current density and time of exposure, the density of pores and their size can be controlled and can be monitored by scanning electron micrography and/or nitrogen adsorption desorption volumetric isothermic measurement. If the particles are porous, their total surface area will be increased by virtue of their porosity. For example, their surface area may be increased by at least about 50% or at least about 100%, compared to the surface area of a corresponding non-porous particle. According to certain embodiments the porosity is at least 30, 40, 50 or 60%. This means that, respectively, 30, 40, 50 or 60% of the particle volume is pore space. Preferred pore diameters range from about 1 nm to about 50 nm, for example from about 5 nm to about 25 nm, for example from about 1 nm to about 5 nm.

The hydrolysable silicon of the fifth or sixth aspects may be or comprise hydrolysable doped silicon. As used herein, the term “doped silicon” may refer to silicon which behaves as an extrinsic semiconductor due to the presence of dopant atoms. The dopant atoms may be or comprise dopant atoms which are substitutional (taking the place of Si atoms). Additionally or alternatively, the dopant atoms may be or comprise dopant atoms which are interstitial (amongst, not displacing, Si atoms).

The silicon may optionally be n-doped or p-doped. The invention includes embodiments wherein the silicon is doped with one or more elements selected from B, P, Mg, Ca, Cu, Ga, Al, In, Bi, Ge, Li, Xe, N, Au and Pt. The dopant may be an n-dopant, for example phosphorus. Most preferably the dopant is a p-dopant. Most preferably the dopant is boron. Use of a p-dopant is especially suitable for stabilising negatively charged nucleic acid.

The manufacture of doped-silicon is well understood in the semiconductor industry and includes ion implantation and diffusion methods. Alternatively, silicon can be doped by using a diffusion method to increase the amount of dopant present in the silicon. As an example, of a diffusion method, silicon powder and a doping reagent (for example B₂O₃ for boron doping) is placed in a bowl which is mixed and placed under an N₂ atmosphere and subjected to a temperature of between 1050° C. and 1175° C. for a few minutes to allow the dopant (for example boron) to diffuse into the silicon.

According to certain embodiments, doping of the silicon is heavy. Heavy boron doping is especially preferred. Heavy doping is understood to mean doping of at least 1×10¹⁵ dopant atoms per cm³. Doping levels correspond to a certain resistivity. For example, when boron is used as the dopant, as is preferred, a doping level of 1×10²⁰ dopant atoms per cm³ corresponds to a resistivity of about 1 mohm-cm. For example, there may be boron present at levels of at least about 1×10¹⁶ boron atoms per cm³ and up to about 1×10²⁰ boron atoms per cm³.

Advantageously, doping the hydrolysable silicon may have a beneficial effect on the electrostatic behaviour of the silicon. Doping may provide for improved loading of an siRNA or variant thereof into a complex as disclosed herein. Without being bound by theory, it is thought that this may help stabilise RNA while it is in circulation in vivo, thus increasing the half-life in vivo of the RNA until it reaches a target cell, where it is subsequently released. Thus, more RNA may reach a target cell in a given time period after administration, compared to when undoped particles are used, leading to more efficient RNA delivery. It will be appreciated that the term “half-life in vivo of the RNA”, as used herein, may refer to the elimination half-life in vivo of the RNA, i.e. the time period taken for the amount of the RNA, once administered, to reduce by about half. It will also be appreciated that the term “amount of the RNA” may refer to the amount of the RNA or a derivative thereof having the same or substantially the same intended pharmaceutical effect.

Doping with a p-dopant (e.g. boron) may lead to improved binding with a negatively charged RNA, especially an RNA having a net negative charge at a pH of about 7.4 (because this is a typical physiological pH), such as an siRNA or variant thereof as disclosed herein.

Where silicon is referred to herein as “undoped” (such as the particles of composition SIS0012 it may mean that no or only small amounts of dopant atoms are present, for example at most about 1×10² dopant atoms per cm³. Additionally or alternatively, “undoped” silicon may mean silicon that does not behave as an extrinsic semiconductor.

Preferably, the ratio of silicon to nucleic acid is between 0.01:1 and 1:8, for example between 1:1 and 1:6, 1:1 and 1:5, 1:1 and 1:4, or between 1:1 and 1:3. Preferably, the ratio of silicon to nucleic acid is between 1:1 and 1:3. Advantageously, this ratio of silicon to nucleic acid further affects the rate of release of, and stabilises, the nucleic acid conveyed by the particle.

At least 30%, for example at least 50%, for example at least 80%, for example at least 90% of the nucleic acid (for example mRNA or pDNA, or an siRNA or variant thereof as disclosed herein) by weight present is associated with the silicon particles. By this, it is meant that it is non-covalently associated with the silicon. Without wishing to be bound by theory, it is hypothesised that when this takes place, the random movement (Brownian motion) of the nucleic acid is reduced and opportunities for it to be degraded are reduced.

The term “lipid”, as used herein, is understood to include fatty acids and fatty acid derivatives, glycerolipids, glycerophospholipids, sphingolipids, saccharolipids and polyketides. The term “lipid” may encompass lipidated oligopeptide (a term used interchangeably herein with the term lipopeptide) wherein a short peptide sequence is conjugated to one or more fatty acid chains. The one or more lipids may therefore comprise one or more lipidated oligopeptides.

The one or more lipids may comprise an ionisable lipid, a cationic lipid (e.g. DOTAP); a helper lipid, e.g. a phospholipid (e.g. DOPE); a structural lipid, e.g. a cholesterol-based lipid; and/or a polyethylene glycol (PEG) lipid (e.g. DSPE-PEG₂₀₀₀).

The one or more lipids may include one or more of: phosphatidylcholine (PC); hydrogenated PC; stearylamine (SA); dioleoylphosphatidylethanolamine (DOPE); cholesteryl 3β-N-(dimethylaminoethyl) carbamate hydrochloride (DC-chol); 1,2-dioleoyl-3-trimethylammonium-propane (DOTAP); PEGylated 1,2-Distearoyl-sn-glycero-3-phosphoethanolamine (DSPE), such as DSPE-PEG₂₀₀₀; and derivatives of any thereof. In certain embodiments, the lipids comprise or consist of DOTAP, DOPE and DSPE-PEG₂₀₀₀.

It has been found that surface treating the particle with a lipid aids in controlling the rate of release of the nucleic acid. The type of lipid used to treat the surface of the nanoparticle may affect the rate of release of the nucleic acid. In particular, surface treating a silicon particle with a lipid has a beneficial effect on the surface charge of the silicon particles, providing them with the requisite zeta potential to allow for improved loading of nucleic acid, and controlling the rate of nucleic acid release at a target site. The presence of the at least one lipid allows for the rate of hydrolysis of the silicon to be controlled, such that the silicon hydrolyses to the bioavailable orthosilicic acid (OSA) degradation product rather than insoluble polymeric hydrolysis products. Controlling the rate of nucleic acid release may advantageously modulate the length of the time period during which protection of the nucleic acid is sustained, especially concerning protection in vivo in the presence of various bodily fluids. For example, more nucleic acid may be delivered to a target cell in a given time period, than for an otherwise identical composition.

In certain embodiments, the lipid or lipids may have an average molecular weight in the range of from 500 to 1000 (for example, when the lipid contains one or more of a cationic lipid (for example, DTDTMA (ditetradecyl trimethyl ammonium), DOTMA (2,3-dioleyloxypropyl-1-trimentyl ammonium), DHDTMA (dihexadecyl trimethyl ammonium), DOTAP (1,2-dioleoyl-3-trimethylammonium propane), a helper lipid, a structural lipid and a PEG lipid, or is selected from one or more of PC, hydrogenated PC, SA, DOPE, DOTAP, DTDTMA, DHDTMA, DC-cholesterol, and derivatives thereof). The ratio of lipid (i.e. total lipid components) to silicon, before any extrusion of filtration process takes place, may be between 1:1 and 40:1, for example 1:1 and 20:1, for example between 1:1 and 18:1, 1:1 and 16:1, 1:1 and 11:1, 1:1 and 10:1, 1:1 and 9:1, 1:1 and 8:1, 1:1 and 13:1, 2:1 and 12:1, 2:1 and 11:1, 2:1 and 10:1, 2:1 and 9:1, 2:1 and 8:1, for example between 1:1 and 7:1, between 2:1 and 7:1, between 3:1 and 6:1, between 4:1 and 5:1.

As described herein, the one or more lipids may be or comprise one or more structural lipids (e.g. a cholesterol-based lipid). However, the one or more lipids may optionally exclude structural lipid. Thus, the one or more lipids may exclude sterols; especially, they may exclude cholesterol. It has been found that the presently disclosed compositions need not rely on these types of lipid, which traditional RNA delivery systems typically rely on. Thus, the compositions disclosed herein have the potential to provide alternatives to RNA delivery systems reliant on these types of lipids, especially cholesterol. Where cholesterol is not available or where its use is otherwise not possible (e.g., due to its effect in the body) this may be advantageous.

As described herein, the one or more lipids may comprise one or more lipidated oligopeptides. Preferably, the one or more lipidated oligopeptides each comprise a fatty acid chain having in the range of about 12 to about 18 carbon atoms. Preferably, the one or more lipidated oligopeptides each comprise 3 to 20 amino acid residues. Thus, the lipidated oligopeptide may be a lipidated tetrapeptide, lipidated pentapeptide or lipidated hexapeptide.

The lipid or lipids component may, in some embodiments, be or comprise a phospholipid. The term “phospholipid” refers to a lipid comprising a fatty acid chain and a phosphate group. Particularly suitable phospholipids are glycerophospholipids. Particularly suitable phospholipids are those in which the polar head group is linked to quaternary ammonium moieties, such as phosphatidylcholine (PC) or hydrogenated phosphatidylcholine. Another example of a phospholipid is DOPE (phosphatidyl ethanolamine or 1,2-dioleoyl-sn-glycero-3-phosphoethanolamine).

Preferably, the side chain(s) of the phospholipid is/are (an) aliphatic side chain(s) with 15 or more carbon atoms, or an ether side chain with 6 or more repeating ether units, such as a polyethylene glycol or polypropylene glycol chain. Lipids with ether side chains may be referred to as “PEG-lipids” or “PEG-ylated” lipids. The PEG-lipid may be a phospholipid such as 1,2-distearoyl-sn-glycero-3-phosphoethanolamine (DSPE) with a PEG side chain, e.g. DSPE-mPEG2000.

The lipid component may include one or more of phosphatidylcholine (PC), hydrogenated PC, stearylamine (SA), dioleoylphosphatidylethanolamine (DOPE), DOTAP, cholesteryl 3β-N-(dimethylaminoethyl) carbamate hydrochloride (DC)-cholesterol, and derivatives thereof. In certain embodiments the lipid component may consist substantially of phosphatidylcholine, hydrogenated phosphatidylcholine, stearylamine, or combinations thereof.

It has been found that lipid to boron-doped silicon molar ratios of between 0.8:1 and 20:1 are particularly advantageous, for example 1:1, 6:1, 8:1, or 10:1, or 12:1 or 16:1

The lipid or lipids component may, in some embodiments, be or comprise an ionisable lipid. The term “ionisable lipid” refers to lipids having a charge that varies depending on the lipid pKa and the environmental pH, for example, remaining uncharged at physiological pH but becoming protonated (positively charged) at low pH. However, the one or more lipids may optionally exclude ionisable lipid(s). Thus, as described above, the compositions as disclosed herein may advantageously be less dependent on the use of specific novel lipids, including ionisable lipids, which may be subject to technical challenges, supply constraints and intellectual property restrictions on their use.

The lipid or lipids component may, in some embodiments, be or comprise a cationic lipid. The term “cationic lipid” refers to positively charged molecules having a cationic head group attached via a suitable spacer to a hydrophobic tail. Examples include DTDTMA (ditetradecyl trimethyl ammonium), DOTMA (2,3-dioleyloxypropyl-1-trimentyl ammonium), DOTAP, DHDTMA (dihexadecyl trimethyl ammonium) and stearylamine (SA). The positive charge is typically stabilised by a negative counter ion. In certain embodiments, especially in relation to vaccine compositions the cationic lipid is, or comprises DOTAP. DOTAP exists in an S and an R enantiomeric form, and may be present as the S-, R-form or as a racemate. Optionally, of the total DOTAP present by weight, the R and S forms may be in approximately equal amounts (i.e. no more than about 60% of the total DOTAP present by weight, of either form). For example, at least about 80, 90, 95, 98, or 99% of total DOTAP is in the R-form. For example, at least about 80, 90, 95, 98, or 99% of total DOTAP is in the S-form.

In certain embodiments, the one or more lipids may optionally comprise or consist essentially of a combination of DOTAP, DOPE and a PEG-lipid (especially DSPE-PEG2000). In certain embodiments, the one or more lipids may optionally comprise or consist essentially of a combination of DOTAP and DOPE. The ratio by weight of DOTAP:DOPE may be in a range of from about 1:2 to about 2:1; such, for example, as about 1:1. The ratio by weight of DOTAP:PEG-lipid may be in a range of from about 10:1 to about 5:1; such, for example, as about 7:1. The ratio by weight of DOPE:PEG-lipid may be in a range of from about 10:1 to about 5:1; such, for example, as about 7:1.

Nonetheless, as described herein, doping of the silicon may enable less cationic lipid, such as DOTAP, to be used, compared to conventional compositions for RNA delivery (such as lipid nanoparticles, LNPs, which comprise cationic lipid). While cationic lipids may be suitable for electrostatic loading of polyanionic nucleic acids (including various form of RNA) their amine-rich nature may cause cytotoxicity, immunogenicity, and non-specific tissue accumulation.

Thus, in an embodiment, the one or more lipids may optionally exclude cationic lipid. As described herein, cationic lipid may not be necessary; especially when doped silicon, more especially p-doped silicon, is used.

Overall, the compositions disclosed herein provide the potential to use less lipid (especially less cationic lipid, such as less DOTAP, and/or less ionisable lipid) in RNA delivery vehicles, compared to conventional RNA delivery vehicles which do not contain hydrolysable silicon particles (e.g. conventional liposomal nucleic acid delivery vehicles, such as those typically used for mRNA delivery in vivo). Additionally or alternatively, the hydrolysable silicon particles can provide the potential for RNA delivery vehicles to be formulated with a wider range of lipids while still providing transfection efficiency, storage stability, and/or targeted delivery to a particular type of tissue, or to a particular type of cell. In turn, this may lead to reduced reliance in the field on specific lipids, particularly cationic lipids, especially cationic lipids which are formulated specifically for the purpose of RNA delivery and which may therefore not be cost-effective or easily accessible.

As described herein, the presence of one or more lipids in the complex according to the sixth aspect of the invention is optional. Thus, in some embodiments, there is no lipid in the complex according to the sixth aspect of the invention. As described hereinabove, the compositions disclosed herein provide the potential to use less lipid and thus may provide the potential to use no lipid at all in the delivery of siRNA.

Optionally, in addition to or instead of the one or more lipids, the particle of the complex according to the sixth aspect of the invention may comprise one or more non-lipid stabilisers for the siRNA, for example quercetin, tyrosine, nicotinamide adenine dinucleotide (NAD) or derivatives thereof.

Optionally, in addition to or instead of the one or more lipids, the particle of the complex according to the sixth aspect of the invention may comprise a peptide containing a cell surface receptor-(for example, integrin-) recognition sequence that confers a degree of cell specificity for in vivo delivery of the siRNA. The peptide may have a “head group” containing a cell surface receptor recognition sequence and additionally a “tail” that can bind non-covalently to the siRNA and/or the silicon.

Optionally, in addition to or instead of the one or more lipids, the particle of the complex according to the sixth aspect of the invention may comprise a polycationic nucleic acid-binding component. The term “polycationic nucleic acid-binding component” is well known in the art and may refer to polymers having at least 3 repeats of cationic amino acid residues or other cationic unit bearing positively charged groups, such polymers being capable of complexion with a nucleic acid under physiological conditions. An example of a nucleic acid-binding polycationic molecule is an oligopeptide comprising one or more cationic amino acids. A polycationic nucleic acid-binding component may, for example, be an oligo-lysine molecule, an oligo-histidine molecule, an oligo-arginine molecule, an oligo-ornithine molecule, an oligo-diaminopropionic acid molecule, an oligo-diaminobutyric acid molecule, or a combined oligomer comprising or consisting of any combination of histidine, arginine, lysine, ornithine diaminopropionic acid, and diaminobutyric acid residues. Further examples of polycationic components include dendrimers and polyethylenimine.

Treating the lipid-treated silicon particles with an amino acid can also provide a beneficial stabilising effect on the nucleic acid. Treating lipid-treated silicon particles with amino acids has been shown to stabilise nucleic acids in biological fluids, for example in ocular tissues and plasma and tissue fluid. Lipid-treated particles formulated with an amino acid in this manner may be particularly suitable for delivery to the body, for example delivery by transcutaneous injection.

In an embodiment, the complex according to the sixth aspect of the invention may additionally comprise one or more amino acids.

In its broadest sense, the term “amino acid” encompasses any artificial or naturally occurring organic compound containing an amine (—NH₂) and carboxyl (—COOH) functional group. It includes α, β, γ and δ amino acids. It includes an amino acid in any chiral configuration. The amino acid may, especially, be a naturally occurring α amino acid. It may be a proteinogenic amino acid or a non-proteinogenic amino acid (such as carnitine, levothyroxine, hydroxyproline, ornithine or citrulline).

The one or more amino acids may help stabilise the silicon particles themselves. In vivo, the one or more amino acids may help to modulate the rate of hydrolysis of the doped silicon, such that the silicon hydrolyses to bioavailable orthosilicic acid (OSA) degradation product; rather than insoluble polymeric hydrolysis products. In this way, the one or more amino acids may complement the function of the one or more lipids of the present disclosure. Controlling the rate of hydrolysis of the silicon may influence the rate of release of API (e.g. a siRNA or variant thereof as disclosed herein) associated with the silicon. Controlling the rate of API release may modulate the length of the time period during which protection of the API is sustained, especially concerning protection in vivo in the presence of various bodily fluids. Thus, more API may be delivered to a target cell in a given time period, than for an otherwise identical composition.

In an embodiment, the amino acid is glycine.

Additionally or alternatively, amino acids which are neutral or positively charged at physiological pH (about pH 7.4), such as tyrosine or arginine, may stabilise negatively charged APIs (e.g. nucleic acids such as a siRNA or derivative thereof as disclosed herein). Meanwhile, amino acids which are neutral or negatively charged at physiological pH (about pH 7.4) may stabilise positively charged APIs. Nonetheless, the interplay of charge-based and/or other interactions (for example, steric interactions) resulting from the combination of doped Si, lipid(s) and amino acid(s) may be such that amino acid(s) which are positively charged at physiological pH may help to stabilise positively charged APIs, or amino acid(s) which are negatively charged at physiological pH may help to stabilise negatively charged APIs.

The ratio by weight of the one or more lipids (i.e. total lipid components) to the amino acid(s) may be in a range of from about 40:1 to about 1:1; such, for example, as about 32:1.

Additionally or alternatively, the complex according to this fifth or sixth aspect of the invention may comprise one or more non-reducing disaccharides, especially trehalose, The ratio by weight of the one or more lipids (i.e. total lipid components) to non-reducing disaccharide may be in a range of from about 20:1 to about 1:1; such, for example, as about 16:1.

In a seventh aspect of the present invention, there is provided a complex comprising:
(i) a particle comprising hydrolysable silicon;
(ii) optionally, one or more lipids; and
(iii) an siRNA which targets an mRNA encoding a mutant FGFR3 protein, wherein said siRNA targets a portion of the mRNA sequence which includes nucleotides encoding a Gly to Arg mutation at position 380 of the protein, and wherein said siRNA is associated with said particle.

In an embodiment of this aspect, the sequence of said siRNA corresponds exactly to the portion of the mRNA sequence which includes nucleotides encoding the Gly to Arg mutation at position 380 of the protein, or the sequence of said siRNA contains one nucleotide difference with the portion of the mRNA sequence which includes nucleotides encoding the Gly to Arg mutation at position 380 of the protein, as long as said one nucleotide difference is not one of the nucleotides which encode the Gly to Arg mutation at position 380 of the protein.

In further embodiments of this aspect, said siRNA, said particle comprising hydrolysable silicon and said lipid (if present) are as defined elsewhere herein.

### Medical Uses

The siRNA molecules, pharmaceutical compositions and complexes as disclosed herein find use in the medical field for treating and or preventing diseases or disorders linked to the G380R mutation in FGFR3, particularly diseases or disorders caused by mutations which result in the expression of aberrant gain of function mutant FGFR3 protein. Accordingly, in an eighth aspect of the invention, there is provided an siRNA molecule or variant thereof, a pharmaceutical composition, or a complex as disclosed herein, for use as a medicament.

According to a ninth aspect of the invention, there is provided an siRNA molecule or variant thereof, a pharmaceutical composition, or a complex as disclosed herein, for use in the treatment of achondroplasia.

In an embodiment of this aspect, said siRNA molecule or variant thereof, pharmaceutical composition, or complex for use as disclosed herein is administered to a human subject before the age of puberty, e.g. a human aged between 1 to 12 years old, for example 1 to 10 years old, 1 to 9 years old, 2 to 8 years old, or 2 to 7 years old. In another embodiment of this aspect, the human subject, e.g. a human aged between 1 to 12 years old, is treated with said siRNA molecule or variant thereof, pharmaceutical composition, or complex for a total duration of 1 month to 7 years, for example 2 months to 6 years, for example 3 months to 5 years, for example 4 months to 3 years, for example 6 months, 1 year, 2 years, or 3 years. For example, a human subject as defined herein may be administered an siRNA or variant thereof as disclosed herein once per month over a period of months or years, for example for 6 months or for 1, 2, 3, 4, 5, 6, 7, 8, 9, 10, 11 or 12 years.

According to a tenth aspect of the invention, there is provided an siRNA molecule or variant thereof, a pharmaceutical composition, or a complex as disclosed herein, for use in downregulating the expression of FGFR3 protein carrying a Gly to Arg mutation at position 380 (G380R).

In an embodiment of this aspect, said siRNA molecule or variant thereof, pharmaceutical composition, or complex for use as disclosed herein is administered to a human subject before the age of puberty, e.g. a human aged between 1 to 12 years old, for example 1 to 10 years old, 1 to 9 years old, 2 to 8 years old, or 2 to 7 years old. In another embodiment of this aspect, the human subject, e.g. a human aged between 1 to 12 years old, is treated with said siRNA molecule or variant thereof, pharmaceutical composition, or complex for a total duration of 0.5 to 7 years, for example 1 to 6 years, for example 1 to 5 years, for example 1 to 3 years, for example 1, 2 or 3 years.

According to an eleventh aspect of the invention, there is provided a method of treating achondroplasia in a mammal in need thereof, comprising administering to said mammal a pharmaceutically effective dose of an siRNA molecule or variant thereof, a pharmaceutical composition, or a complex as disclosed herein.

In an embodiment of this aspect, the mammal is a human. In another embodiment of this aspect, the mammal is before the age of puberty, e.g. a human aged between 1 to 12 years old, for example 1 to 10 years old, 1 to 9 years old, 2 to 8 years old, or 2 to 7 years old. In another embodiment of this aspect, the human, e.g. a human aged between 1 to 12 years old, is treated with said siRNA molecule or variant thereof, pharmaceutical composition, or complex for a total duration of 0.5 to 7 years, for example 1 to 6 years, for example 1 to 5 years, for example 1 to 3 years, for example 1, 2 or 3 years.

The amount of the siRNA molecule or variant thereof, pharmaceutical composition, or complex as disclosed herein which is required to achieve a therapeutic effect will vary with the particular route of administration and the characteristics of the subject under treatment (for example the age, weight, sex, or other concurrent medical conditions) and can be readily determined and administered by an ordinarily skilled physician.

The siRNA molecule or variant thereof, pharmaceutical composition, or complex as disclosed herein may be administered by systemic injection or by localised administration, e.g. to mucous membranes. Advantageously, the siRNA molecule or variant thereof, pharmaceutical composition, or complex is administered systemically, for example parenterally, for example intravenously.

Optimisation of the siRNA Molecules

siRNA molecules as described herein may be further optimised for in vivo use via various chemical modifications. Such chemical modifications may for example improve stability of the RNA duplex, further reduce the likelihood of off-target effects, improve pharmacodynamics, and/or decrease immunogenicity. Therefore, one or more of the nucleotides in an siRNA molecule or variant thereof as disclosed herein may be chemically modified, for example at the 2′ position of the ribose ring, for example via a 2′-alcoxy, 2′-methoxy, 2′-ethoxy, 2′-fluoro, 2′-O-(2-methoxyethyl), 2′-O-benzyl, 2′-O-methyl-4-pyridinil, 2′-amino, 2′-aminoethyl, or 2′-guanidinopropyl modification.

An siRNA or variant thereof as disclosed herein may also be combined with carriers, for example polymer carriers, to optimise siRNA delivery to target cells. The siRNA molecules or variants thereof as disclosed herein may therefore be bound to or complexed with a carrier, for example a polymer carrier, for example polyethyleneimine (PEI) or a derivative thereof (such as polyethyleneimine-polyethylene-glycol-N-acetylgalactosamine (PEI-PEG-GAL or polyethyleneimine-polyethylene glycol-tri-N-acetyl galactosamine (PEI-PEG-triGAL)).

The phosphate backbone of an siRNA or variant thereof as disclosed herein may be modified in order to increase resistance to nuclease degradation. This may take place at the 5′ or 3′ end (to increase resistance to exonucleases) or throughout the entire siRNA molecule (increasing resistance to endonucleases). Such modifications include phosphorothioate linkages, which substitute a sulfur atom for a non-bridging oxygen in the phosphate backbone. The siRNA molecules or variants thereof as disclosed herein may therefore comprise 1 or more phosphorothioate linkages.

An siRNA or variant thereof as disclosed herein may contain different types of modified nucleotides. The siRNA molecules or variants thereof as disclosed herein may comprise one or more modified nucleotides, for example one or more modified nucleotides independently selected from the group consisting of 2′-fluoro, 2′-amino, 2′-thio and 2′-deoxy modified ribonucleotides. Said one or more modified nucleotides may be present in the sense strand or the antisense strand, for example a 2′-fluoro modified ribonucleotide that is present within the sense strand.

The siRNA molecules or variants thereof as disclosed herein may for example comprise one or more modified nucleotides, for example one or more modified nucleotides independently selected from the group consisting of 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine and 2′-amino-butyryl-pyrene-uridine.

The siRNA molecules or variants thereof as disclosed herein may for example comprise a modified nucleotide selected from the group consisting of 5-bromo-uridine, 5-iodo-uridine, 5-methyl-cytidine, ribo-thymidine, 2-aminopurine, 5-fluoro-cytidine, and 5-fluoro-uridine, 2,6-diaminopurine, 4-thio-uridine; and 5-amino-allyl-uridine.

The siRNA molecules or variants thereof as disclosed herein may for example comprise one or more 2′-O-methyl (2′OMe) modifications, wherein a methyl group is added to the 2′ hydroxyl of the ribose moiety, thus forming 2′OMe-guanosine, 2′OMe-uridine, 2′OMe-adenosine or 2′OMe cytosine. Such modifications may occur independently at one or more of the positions of the siRNA molecule.

siRNA molecules may also be modified by replacing ribose with different sugar moieties, for example 6-carbon moieties such as glucose. Therefore, siRNA molecules or variants thereof as disclosed herein may be modified by substituting different moieties for ribose, for example galactose, mannose, dextrose, glucose, sucrose, or trehalose moieties.

siRNAs may be conjugated to various moieties via terminal or non-terminal nucleotides. siRNA molecules or variants thereof as disclosed herein may therefore be conjugated to moieties such as N-acetylgalactosamine, N-Acetylcysteine, N-Acetylaspartate, N-Acetyltyrosine, or N-Acetylprocainamide, via any terminal or non-terminal nucleotide.

The invention will now be described further with respect to the following experimental examples.

### EXAMPLES

### Example 1: In Silico Design of FGFR3^{G380R}-Specific siRNAs and Primer Pairs

Candidate siRNA sequences specific for the mutant FGFR3^{G380R} mRNA were identified and then analysed using in silico techniques. The coding DNA sequences which were used as the basis for analysing siRNA sequences are shown in the Table 1 below.

TABLE 1

| | | | Coding | Coding | | |
|---|---|---|---|---|---|---|
| | | | DNA Seq | DNA Seq | | |
| GenBank Acc# | Description | Length | Start | End | Type | Set |
| — | Mutation c. | 4301 | 276 | 2696 | Ref | Hum_mut |
| | G1138A in | | | | | |
| | Homo sapiens | | | | | |
| | FGFR3, | | | | | |
| | transcript | | | | | |
| | variant 1, | | | | | |
| | mRNA. | | | | | |
| NM_000142 | Homo sapiens | 4301 | 276 | 2696 | match | Hum_wt1 |
| | FGFR3 transcript | | | | | |
| | variant 1, | | | | | |
| | mRNA. | | | | | |
| NM_001163213 | Homo sapiens | 4307 | 276 | 2702 | match | Hum_wt |
| | FGFR3 transcript | | | | | |
| | variant 3, | | | | | |
| | mRNA. | | | | | |
| NM_022965 | Homo sapiens | 3965 | 276 | 2360 | match | Hum_wt |
| | FGFR3 transcript | | | | | |
| | variant 2, | | | | | |
| | mRNA. | | | | | |
| NM_001354810 | Homo sapiens | 4233 | 276 | 2654 | match | Hum_wt |
| | FGFR3, | | | | | |
| | transcript | | | | | |
| | variant 5, | | | | | |
| | mRNA. | | | | | |
| NM_001354809 | Homo sapiens | 4304 | 276 | 2699 | match | Hum_wt |
| | FGFR3 transcript | | | | | |
| | variant 4, | | | | | |
| | mRNA. | | | | | |
| NM_001359036 | Mus musculus | 4248 | 335 | 2740 | match | Mus_wt |
| | Fgfr3 transcript | | | | | |
| | variant 6, | | | | | |
| | mRNA. | | | | | |
| NM_001359037 | Mus musculus | 3964 | 510 | 2456 | match | Mus_wt |
| | Fgfr3 transcript | | | | | |
| | variant 7, | | | | | |
| | mRNA. | | | | | |
| NM_001163217 | Mus musculus | 4020 | 104 | 2512 | match | Mus_wt |
| | Fgfr3 transcript | | | | | |
| | variant 4, | | | | | |
| | mRNA. | | | | | |
| NM_001163216 | Mus musculus | 3960 | 104 | 2452 | match | Mus_wt |
| | Fgfr3 transcript | | | | | |
| | variant 3, | | | | | |
| | mRNA. | | | | | |
| NM_001205270 | Mus musculus | 4222 | 309 | 2714 | match | Mus_wt |
| | Fgfr3 transcript | | | | | |
| | variant 5, | | | | | |
| | mRNA. | | | | | |
| NM_008010 | Mus musculus | 4219 | 309 | 2711 | match | Mus_wt |
| | Fgfr3 transcript | | | | | |
| | variant 1, | | | | | |
| | mRNA. | | | | | |
| NM_001163215 | Mus musculus | 4245 | 335 | 2737 | match | Mus_wt |
| | Fgfr3 transcript | | | | | |
| | variant 2, | | | | | |
| | mRNA. | | | | | |

The first sequence in the table is a modified NM_000142 sequence with the single G1138A mutation inserted. The column entitled “Set” indicates which set the wildtype transcripts were grouped in for homology analysis. The wildtype NM_000142 sequence was placed into a separate “Hum_wt1” set so that it could be directly compared to the mutant transcript version. The remaining human NM sequences were grouped into a separate set and all of the mouse NM sequences were grouped as a fourth set.

Starting from the mutant FGFR3^{G380R} coding DNA sequence, various siRNAs were designed to complement the point mutation region in the mRNA of the mutated human FGFR3 gene. All siRNAs included the single nucleotide substitution which is responsible for the ACH phenotype (i.e. the G1138A mutation in the human FGFR3 gene). To increase the siRNA specificity, the siRNA design strategy optionally included an additional nucleotide mismatch compared to the corresponding target sequence of the mutant FGFR3 mRNA (i.e. a maximum of 2 mismatches compared to the corresponding wild-type mRNA).

The designed siRNA molecules were then analysed in silico. Key criteria used for analysis included a calculation of the average adjusted number of mismatches across all alternative transcripts in a particular sequence set (see Table 1 and the paragraph following Table 1) within the core (central) 8 nt of the sequence, as well as a calculation of the maximum net free energy difference between the siRNA antisense sequence binding to all the intended target context sequences (usually the human gene) compared to the stated sequence set (mouse NM, etc.), all under standard physiological salt conditions. siRNA molecules which had either the biggest difference in AG between Hum_mut and Hum_wt1 sets, or had >=0.5 mismatches between these two sets in the central 8 nt core of the siRNA were considered to have favourable characteristics. Additional criteria which were used for analysing potential siRNA sequences included:
oligo monomer AG: the calculated folding free energy for the siRNA folding on itself;
target monomer AG: the calculated folding free energy for the target context sequence folding on itself;
oligo homodimer AG: the calculated binding free energy for the siRNA binding to another copy of itself;
heterodimer net AG: the net binding free energy for the binding of the antisense siRNA to the target context sequence while accounting for competition with the other folded structures;
the number of mismatches between the sense siRNA (excluding the TT overhangs) and the closest matching human wildtype sequence without factoring in bulges and frame shifts;
the percent of the full length siRNA sequence that is either G or C;
predicted binding to homologous sequences in the mutant and wild-type genes from human as well as the wildtype mouse transcripts (with good homology to the mutant but poor homology to other sequences being preferred).

Thirty candidate siRNA sequences were identified and are shown in Table 2 below. Primer pairs specific for the mutant FGFR3^{G380R} mRNA were also designed (Table 3). These are useful for gene expression analysis (an essential tool to monitor the gene downregulation induced by the siRNA treatment) while removing the confounding effect of wild-type FGFR3 expression.

TABLE 2
Candidate siRNA sequences. Nucleotides in bold
indicate the G1138A mutation; underlined
nucleotides indicate additional mismatches
relative to the wild-type FGFR3 sequence.

| | Total no. | | |
|---|---|---|---|
| | of mis- | | |
| | matches | | |
| | wrt wt | | SEQ |
| | FGFR3 | | ID |
| Name | sequence | Sequence | NO. |
| G380R_1 | 1 | GCAGGCAUCCUCAGCUACA | 15 |
| G380R_2 | 2 | GCAGGCAUCCUCAGCUAUA | 16 |
| G380R_3 | 2 | GCAGGCAUCCUCAGGUACA | 17 |
| G380R_4 | 2 | GCAGGCAUCCUCUGCUACA | 18 |
| G380R_5 | 1 | GCUACAGGGUGGGCUUCUU | 19 |
| G380R_6 | 2 | GCUAUAGGGUGGGCUUCUU | 3 |
| G380R_7 | 2 | GCUACAGGGUGGCCUUCUU | 20 |
| G380R_8 | 2 | GCUACAGGGUGGGCAUCUU | 4 |
| G380R_9 | 1 | AGGGUGGGCUUCUUCCUGU | 21 |
| G380R_10 | 2 | AAGGUGGGCUUCUUCCUGU | 6 |
| G380R_11 | 2 | AGGGUGGGCUUCUUUCUGU | 7 |
| G380R_12 | 2 | AGGGUGGGCUUCAUCCUGU | 22 |
| G380R_13 | 1 | UCAGCUACAGGGUGGGCUU | 23 |
| G380R_14 | 2 | UCAGCUAUAGGGUGGGCUU | 24 |
| G380R_15 | 2 | UCAGCUACAGGGUCGGCUU | 9 |
| G380R_16 | 2 | UCAGCUACAGGGAGGGCUU | 10 |
| G380R_17 | 1 | GCAUCCUCAGCUACAGGGU | 25 |
| G380R_18 | 2 | GCAUCCUCAGCUAUAGGGU | 12 |
| G380R_19 | 2 | GCAUCCUCAGCUUCAGGGU | 26 |
| G380R_20 | 2 | GCAUCCUCAGCCACAGGGU | 27 |
| G380R_21 | 2 | GUAGGCAUCCUCAGCUACA | 28 |
| G380R_22 | 2 | GCAGGUAUCCUCAGCUACA | 29 |
| G380R_23 | 2 | GUUACAGGGUGGGCUUCUU | 30 |
| G380R_24 | 2 | UCUACAGGGUGGGCUUCUU | 31 |
| G380R_25 | 2 | AGUGUGGGCUUCUUCCUGU | 32 |
| G380R_26 | 2 | UCAUCCUCAGCUACAGGGU | 33 |
| G380R_27 | 2 | GCAUCCUCAGCUACAUGGU | 34 |
| G380R_28 | 2 | GCAUCCUCAGCUACAGGAU | 35 |
| G380R_29 | 2 | ACAGCUACAGGGUGGGCUU | 36 |
| G380R_30 | 2 | UCAGCUACAUGGUGGGCUU | 37 |

TABLE 3
Candidate primer pairs specific for FGFR3^{G380R}
mutant mRNA.

| | Forward/ | | SEQ |
|---|---|---|---|
| Name | Reverse | Primer Sequence | ID NO. |
| G380R_Primer pair 1 | Forward | AGGCATCCTCAGCTACA | 38 |
| G380R_Primer pair 1 | Reverse | GGCAGGCAGCTCGAGCTCGG | 39 |
| G380R_Primer pair 2 | Forward | CTACAGGGTGGGCTTCTTCC | 40 |
| G380R_Primer pair 2 | Reverse | GTGGTGTGTTGGAGCTCATG | 41 |
| G380R_Primer pair 3 | Forward | CAGGCATCCTCAGCTACAGG | 42 |
| G380R_Primer pair 3 | Reverse | TCATGGACGCGTTGGACT | 43 |
| G380R_Primer pair 1- | Forward | Flap | 44 |
| MODF | | AATAAATCATAAAGGCATCC | |
| | | TCAGCTACA | |
| G380R_Primer pair 1- | Reverse | Flap | 45 |
| MODF | | AATAAATCATAAGGCAGGCA | |
| | | GCTCGAGCTCGG | |
| G380R_Primer pair | Forward 1 | GGCATCCTCAGCTACAG | 46 |
| 4F1 | | | |
| G380R_Primer pair | Forward 2 | CAGGCATCCTCAGCTACA | 47 |
| 4F2 | | | |
| G380R_Primer pair 4 | Reverse | CGAGACAGCTCCCATTT | 48 |
| G380R_Primer pair 5 | Forward | TCTCTCCTTGCACAACG | 49 |
| G380R_Primer pair 5- | Reverse 1 | TGTAGCTGAGGATGCCTG | 50 |
| 1 | | | |
| G380R_Primer pair 5- | Reverse 2 | TGTAGCTGAGGATGCCT | 51 |
| 2 | | | |
| G380R_Primer pair | Forward | CATCACTCTGCGTGGCTG | 52 |
| Human FGFR3 F | | | |
| G380R_Primer pair | Reverse | TGTAGCTGAGGATGCCTGC | 53 |
| Human FGFR3 R | | | |
| G380R_Primer pair | Forward 1 | Flap | 54 |
| 4F1MODF | | AATAAATCATAAGGCATCCT | |
| | | CAGCTACAG | |
| G380R_Primer pair | Forward 2 | Flap | 55 |
| 4F2MODF | | AATAAATCATAACAGGCATC | |
| | | CTCAGCTACA | |
| G380R_Primer pair 4- | Reverse | Flap | 56 |
| MODF | | AATAAATCATAACGAGACAG | |
| | | CTCCCATTT | |
| G380R_Primer pair 5- | Forward | Flap | 57 |
| MODF | | AATAAATCATAATCTCTCCTT | |
| | | GCACAACG | |
| G380R_Primer pair 5- | Reverse 1 | Flap | 58 |
| 1MODF | | AATAAATCATAATGTAGCTG | |
| | | AGGATGCCTG | |
| G380R_Primer pair 5- | Reverse 2 | Flap | 59 |
| 2MODF | | AATAAATCATAATGTAGCTG | |
| | | AGGATGCCT | |

### Example 2: Generation of FGFR3^{WT}- and FGFR3^{G380R}-Expressing HEK293 Cells

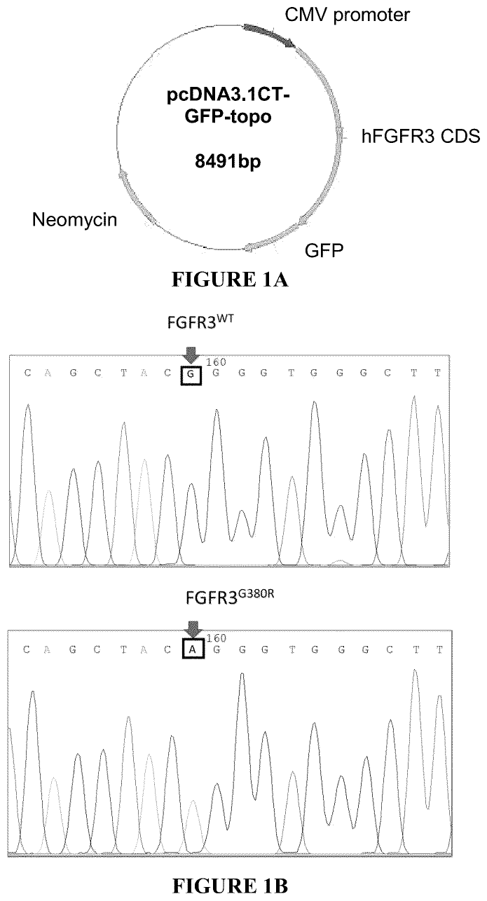

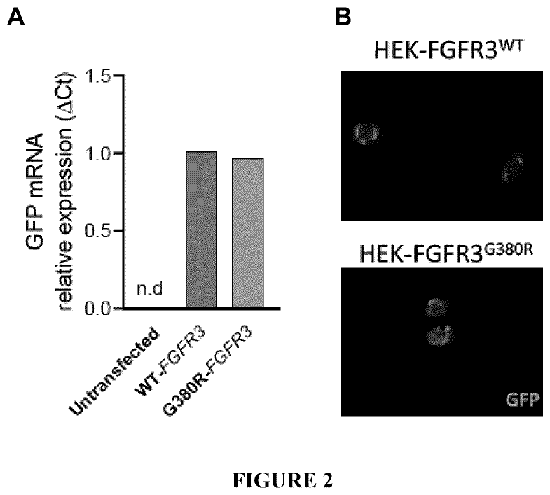

FGFR3^{WT}- and FGFR3^{G380R}-expressing cells for in vitro screening were generated via stable transfection of HEK293 cells with GFP-tagged expression vectors carrying either the wild type (FGFR3^{WT}) or the G380R mutant (FGFR3^{G380R}) FGFR3 gene variant. Briefly, the expression vectors were generated by cloning the human CDS of the wild type FGFR3 gene into the pcDNA3.1/CT-GFP-fusion expression vector containing a resistance cassette for Neomycin (FIG. 1A). The mutant FGFR3^{G380R} expression vector was obtained by site-directed mutagenesis of the wild type FGFR3 CDS. The presence of the target mutation was confirmed by Sanger sequencing (FIG. 1B). After generation, expression vectors were transfected into HEK293 cells using Lipofectamine® 3000 reagent, and transfected cells were positively selected via application of the antibiotic Geneticin (G-418) for 4 weeks. At the end of the selection step, expression of GFP was confirmed at mRNA and protein level by real time RT-PCR (FIG. 2A) and fluorescence microscopy (FIG. 2B), respectively. The results showed that both HEK-FGFR3^{WT} and HEK-FGFR3^{G380R} cells expressed high levels of GFP, further confirming the success of the transfection. Since the expression vectors used generate an FGFR3-GFP fusion protein, the expression of GFP is directly linked with the expression of FGFR3 and can be used to monitor both gene and protein expression, representing a reliable read out for screening of FGFR3^{G380R}-specific siRNAs.

### Example 3: In Vitro Screening of Candidate FGFR3^{G380R}-Specific siRNAs in FGFR3^{WT}- and FGFR3^{G380R}-Expressing HEK293 Cells

Preliminary siRNA screening was performed using the transfected HEK cells generated in Example 2. The screening took into account the efficacy of candidate siRNAs in downregulating the mutant FGFR3^{G380R} mRNA without affecting the expression of wild-type FGFR3 (FGFR3^{WT}). A first subset of 10 siRNAs, from the 30 FGFR3^{G380R}-specific candidate siRNAs identified by the in silico design process of Example 1, was selected for preliminary screening (Table 4).

TABLE 4
Sequences of 10 FGFR3^{G380R} siRNAs screened in vitro. Nucleotides
in bold indicate the G1138A mutation; underlined nucleotides
indicate additional mismatches relative to the wild-type FGFR3
sequence.

| | Total no. of | | |
|---|---|---|---|
| | mismatches | | |
| | wrt wt | | |
| Name | FGFR3 | | SEQ ID |
| (siRNA #) | sequence | Sequence | NO. |
| G380R_6 | 2 | GCUAUAGGGUGGGCUUCUU | 3 |
| (6) | | | |
| G380R_7 | 2 | GCUACAGGGUGGCCUUCUU | 20 |
| (7) | | | |
| G380R_8 | 2 | GCUACAGGGUGGGCAUCUU | 4 |
| (8) | | | |
| G380R_10 | 2 | AAGGUGGGCUUCUUCCUGU | 6 |
| (10) | | | |
| G380R_11 | 2 | AGGGUGGGCUUCUUUCUGU | 7 |
| (11) | | | |
| G380R_12 | 2 | AGGGUGGGCUUCAUCCUGU | 22 |
| (12) | | | |
| G380R_14 | 2 | UCAGCUAUAGGGUGGGCUU | 24 |
| (14) | | | |
| G380R_15 | 2 | UCAGCUACAGGGUCGGCUU | 9 |
| (15) | | | |
| G380R_16 | 2 | UCAGCUACAGGGAGGGCUU | 10 |
| (16) | | | |
| G380R_18 | 2 | GCAUCCUCAGCUAUAGGGU | 12 |
| (18) | | | |

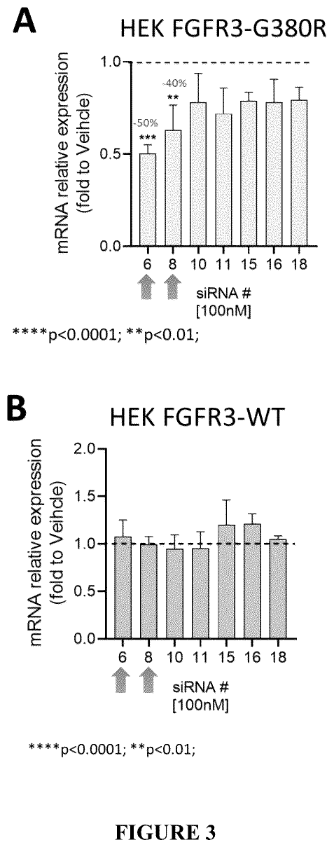

Briefly, both HEK-FGFR3^{WT} and HEK-FGFR3^{G380R} cells were treated with 100 nM of the selected siRNA, combined with the Dharmafect transfection reagent, for 48 hours. Vehicle (Dharmafect)-treated HEK cells were used as control. At the end of the experiment, RNA was isolated, and the efficacy and specificity of the candidate siRNAs was assessed via RT-PCR analysis of GFP mRNA expression. Results are shown in FIG. 3 and in Table 5 below. The results showed that FGFR3^{G380R}-siRNAs number 6 and 8 (SEQ ID NO. 3 and 4, respectively) were particularly effective in downregulating the mutant FGFR3^{G380R} mRNA (−50% and −40%, respectively) (FIG. 3A) without affecting the expression of the wild-type counterpart (FIG. 3B).

TABLE 5
Initial in vitro screening results for candidate FGFR3^{G380R}-specific siRNAs. HEK-FGFR3^{G380R} denotes HEK293 cells expressing
the mutant FGFR3 gene, HEK-FGFR3^{WT} denotes HEK293 cells expressing wild-type FGFR3. Gene expression is expressed in terms
of fold change compared to vehicle control and normalised by human GAPDH. Each value in the table represents an independent
experiment and p-values were calculated using a Multiple Comparison ANOVA (MC-ANOVA) without any correction.

| | HEK FGFR3-G380R | | | | | | | | | |
|---|---|---|---|---|---|---|---|---|---|---|
| siRNA # | 6 | 7 | 8 | 10 | 11 | 12 | 14 | 15 | 16 | 18 |
| Fold change vs | 0.532949 | 0.532949 | 0.679275 | 0.902543 | 0.563336 | 0.836286 | 1.022477 | 0.796678 | 0.684 | 0.743327 |
| Vehicle | 0.521981 | 0.460754 | 0.688758 | 0.877863 | 0.78571 | 0.78571 | 1.022477 | 0.738192 | 0.738192 | 0.76954 |
| | 0.525611 | 0.457571 | 0.723 | 0.78571 | 0.813418 | 0.738192 | 1.051223 | 0.830509 | 0.921508 | 0.871799 |
| | 0.432269 | 0.323088 | 0.429283 | 0.558644 | | | | | | |
| p-value | <0.0001 | <0.0001 | 0.0036 | 0.2498 | 0.0749 | 0.3116 | 0.0523 | 0.3217 | 0.2812 | 0.361 |
| | HEK FGFR3-WT | | | | | | | | | |
| siRNA # | 6 | 7 | 8 | 10 | 11 | 12 | 14 | 15 | 16 | 18 |
| Fold change vs | 1.079407 | 0.534767 | 1.084464 | 1.011841 | 0.83046 | 0.660661 | 1.476297 | 1.015354 | 1.095799 | 1.022416 |
| Vehicle | 0.908767 | 0.651566 | 0.927862 | 1.05116 | 1.154272 | 1.018879 | 1.122708 | 1.496905 | 1.285191 | 1.040288 |
| | 1.250047 | 0.629371 | 0.977374 | 0.777537 | 0.868732 | 0.71548 | 1.363186 | 1.095799 | 1.258742 | 1.088229 |
| p-value | 0.5405 | 0.0053 | 0.9788 | 0.6813 | 0.7058 | 0.1286 | 0.0199 | 0.1268 | 0.1092 | 0.6974 |

### Example 4: In Vitro Testing of Candidate FGFR3^{G380R}-Specific siRNAs complexed with nanoparticles comprising hydrolysable silicon in FGFR3^{G380R}-Expressing HEK293 Cells

Candidate FGFR3^{G380R}-siRNAs number 6 and 8 (SEQ ID NO. 3 and 4, respectively) were selected to undergo further in vitro screening, using the FGFR3^{G380R}- and FGFR3^{WT}-expressing HEK293 cells generated in Example 2. The siRNAs were complexed with silicon stabilised hybrid lipid nanoparticles (sshLNP™) (‘BioCouriers’) manufactured by SiSaf Ltd (Guildford, UK).

Nanoparticles used were SIS0013 and SIS0012 2LBS (formulated with doped material) and non-doped SIS0012. Nanoparticles and siRNA complexes were produced using the protocols described in UK patent application no. 2300912.9 and as outlined below. Briefly, the composition of the nanoparticles was as follows:
SIS0012 (undoped Si) formulation
   DOTAP: 7.25 mg
   DOPE: 7.30 mg
   mPEG₂₀₀₀-DSPE: 1.45 mg
   Si (undoped): 1 mg
   Glycine: 0.5 mg
   Trehalose: 1 mg
   Nuclease-free water: up to 10 mL
SIS0013 (doped Si) formulation
   DOTAP: 7.25 mg
   DOPE: 7.3 mg
   mPEG₂₀₀₀-DSPE: 1.45 mg
   Boron-doped Si, circa 5×10¹⁸ boron atoms/cm³: 1 mg
   Glycine: 0.5 mg
   Trehalose: 1 mg
   Nuclease-free water: up to 10 mL
SIS0012 2LBS (doped Si) formulation
   DOTAP: 7.75 mg
   DOPE: 8.25 mg
   Boron-doped Si, circa 5×10¹⁸ boron atoms/cm³: 1 mg
   Glycine: 0.5 mg
   Trehalose: 1 mg
   Nuclease-free water: up to 10 mL

Additional details of the above-disclosed components of SIS0012, SIS0013 and SIS0012 2LBS are as follows:
a) Si (“SiNPs”): as porous silicon particles of 30 nm average diameter. Doped for SIS0013 and SIS0012 2LBS (5×10¹⁸ boron atoms/cm³). Activated by exposure to methanol followed by slow evaporation, producing (dry, solid) powder, which is activated SiNPs.
b) Trehalose (“THR”): as solid powder.
c) Glycine (“GLY”): as solid powder.
d) SiNPs+GLY+THR solution: as a brownish suspension, obtained via sonication for 60 minutes of a suspension in 50 ml nuclease-free water of: 50 mg activated SiNPs; 50 mg THR; and 25 mg GLY.
e) DOTAP-CI Solution: as a solution obtained by mixing 50 mg DOTAP in 10 ml of methanol, followed by sonication at 40° C. for 30 minutes, until fully dissolved.
f) DOPE Solution: as a solution, obtained by mixing 50 mg DOPE in 10 ml of methanol, followed by sonication at 40° C. for 30 minutes, until fully dissolved.
g) mPEG₂₀₀₀-DSPE Solution: as a solution, obtained by mixing 40 mg of mPEG₂₀₀₀-DSPE in 8 ml of methanol, followed by sonication at 40° C. for 30 minutes, until fully dissolved.

The method for preparing SIS0012, SIS0012-2LBS or SIS0013 complexes was as follows:

### Lipid Film Preparation

a) Mix all lipids (DOPE, DOTAP and mPEG2000-DSPE for SIS0012 and SIS0013; DOPE and DOTAP for SIS0012 2LBS) in a glass round bottomed flask.
b) Evaporate solvent with rotary evaporator in water bath at 40° C.

### Rehydration of the Film

a) Add 1 ml of the brownish suspension of SiNPs (SIS0013 and SIS0012 2LBS: doped; SIS0012: undoped)+GLY+THR (1 mg/ml SiNPs; 0.5 mg/ml GLY; 1 mg/ml THR) to the lipid film, along with 9 ml nuclease free water (total volume of the brownish suspension and nuclease free water together is thus 10 ml).
b) Cover the flask with parafilm, then agitate the flask in a water bath at 60° C. for 10 minutes, thus rehydrating the lipid film by means of the 10 ml of liquid.
c) Leave resultant suspension to rest at room temperature for a few hours before storing at 4° C.

### Extrusion

Pass the suspension obtained in step (c) of “Rehydration of the film” through a polycarbonate membrane filter having 0.4 μm and 0.1 μm pore sizes. Pass the suspension 10 times, at 60° C., through each pore size. The resultant products are SIS0012 where undoped SiNPs are used and SIS0013 or SIS0012 2LBS where doped SiNPs are used.

Preparation of siRNA Complexes

The required volumes of the appropriate SIS0012, SIS0012 2LBS or SIS0013 suspension (with a nominal total lipid concentration of 1.6 mg/mL) were mixed with the required volumes of siRNA stock solution (with concentration of 2 mg/mL) and the final concentration of siRNA was adjusted using nuclease-free water before the complexation incubation step. The samples were mixed thoroughly by gently pipetting and were incubated at room temperature for 60 min to allow for complexation to complete. Following incubation, samples were stored at 4° C. prior to their use in subsequent experiments.

GFP-tagged FGFR3^{G380R}-expressing HEK293 cells generated in Example 2 were seeded at 500K cell density, transfected for 24 hours and evaluated after 48 hours. GFP gene expression was evaluated by real-time PCT, expressed in terms of fold change relative to vehicle control, and normalised by human GAPDH. SIS0013 and SIS0012 2LBS (formulated with doped material) significantly reduced GFP expression when complexed to both candidate FGFR3^{G380R}-siRNA numbers 6 and 8 (SEQ ID NOs 3 and 4) (Table 6).

TABLE 6
In vitro screening results for candidate FGFR3^{G380R}-specific siRNAs complexed to
nanoparticles comprising hydrolysable silicon. 2-way ANOVA statistical analysis
applied, followed by a post hoc Tukey's multiple comparison test.
HEK FGFR3^{G380R}

| | | SIS0012- | SIS0012- | | SIS0013- | SIS0013- | | SIS0012- | SIS0012- |
|---|---|---|---|---|---|---|---|---|---|
| | SIS0012- | 2LBS-siRNA | 2LBS-siRNA | | siRNA 6 | siRNA 8 | | siRNA 6 | siRNA 8 |
| | 2LBS | 6 (SEQ | 8 (SEQ | SIS0013 | (SEQ ID | (SEQ ID | SIS0012 | (SEQ ID | (SEQ ID |
| Complex | empty | ID NO. 3) | ID NO. 4) | empty | NO. 3) | NO. 4) | empty | NO. 3) | NO. 4) |
| Fold | 0.99539 | 0.780967 | 0.764895 | 1.104454 | 0.866537 | 0.802923 | 0.97041 | 1.130269 | 1.004632 |
| Change Vs | 0.968171 | 0.713672 | 0.814131 | 0.897095 | 0.819794 | 0.743979 | 0.963707 | 1.0693 | 1.025741 |
| Vehicle | 1.03766 | 0.814131 | 0.759611 | 1.009285 | 0.808508 | 0.878633 | 1.0693 | 1.254112 | 0.97716 |
| p value | >0.99 | 0.0054 | 0.0081 | >0.99 | 0.0579 | 0.0232 | >0.99 | 0.1307 | >0.99 |

### Example 5: Generation of In Vivo Achondroplasia Mouse Model

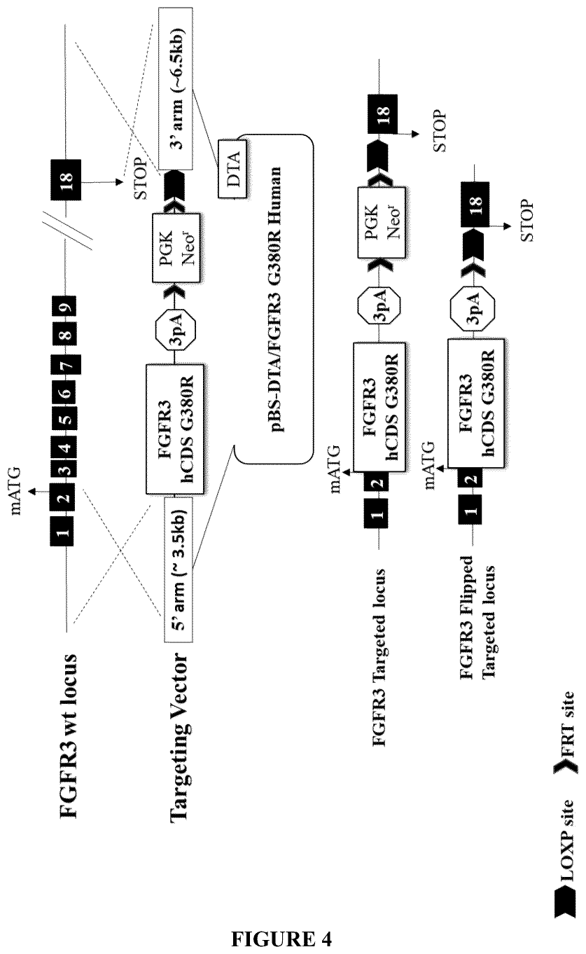

For in vivo testing of the siRNAs, a humanised knock-in (KI) mouse model of ACH is generated carrying the mutant human heterozygous FGFR3 gene encoding the G380R mutation. In this model, the human mutant FGFR3 cDNA (NM_000142.5) encoding the FGFR3^{G380R} protein is used to replace the wild-type allele in the mouse Fgfr3 gene. This mouse model fully recapitulates the human disease and is the best choice for developing a siRNA-based therapeutic approach (Lee Y C, Song I W, Pai Y J et al. (2017), Knock-in (KI) human FGFR3 achondroplasia mutation as a mouse model for human skeletal dysplasia. Sci Rep 7:43220, DOI: 10.1038/srep43220). To generate the KI mouse model, a targeting vector is designed to replace the mouse Fgfr3 gene with the human FGFR3 coding DNA sequence encoding the G380R mutation, using homologous recombination. A schematic of vector design and subsequent homologous recombination is shown in FIG. 4.

This vector is then used to generate mouse embryonic stem cells (ESCs) (strain 129/Sv, agouti) carrying the human FGFR3 coding DNA sequence encoding the G380R mutation. The resulting ESCs are injected into the blastocysts of C57Bl/6J female mice. From the chimaeras thus obtained, 3 males with the highest percentage of chimerism (assessed by coat colour) are selected. These are crossed with 3 wild-type females (B6-FGFR3WT/WT). From the F1 generation produced by this cross, around 6 pups per litter with a transmission of the mutation of 40-50% (F1 generation) are obtained. Males with heterozygous genotypes derived from F1 are crossed with wild-type females until F10 is reached. At each crossing, the desired genotype is selected by genotyping, using digital biopsies collected after weaning. Candidate siRNAs are tested using this in vivo mouse model of ACH and their impact on expression of the mutant FGFR3 allele in vivo and their impact on disease are determined.

### Example 6: Generation and Genotyping Results for In Vivo Achondroplasia Mouse Model (FGFR3^{G380R} KI Mice)

In this model, the human mutant FGFR3 cDNA (NM_000142.5) encoding the FGFR3^{G380R} protein was used to replace the wild-type allele of the mouse Fgfr3 gene. The mouse model was generated based on (Lee Y C, Song I W, Pai Y J et al. (2017), Knock-in human FGFR3 achondroplasia mutation as a mouse model for human skeletal dysplasia. Sci Rep 7:43220, DOI: 10.1038/srep43220), with appropriate modifications. A summary of the approach is set out in Example 5. The model fully recapitulates human disease and is the best choice for developing an siRNA-based therapeutic approach.

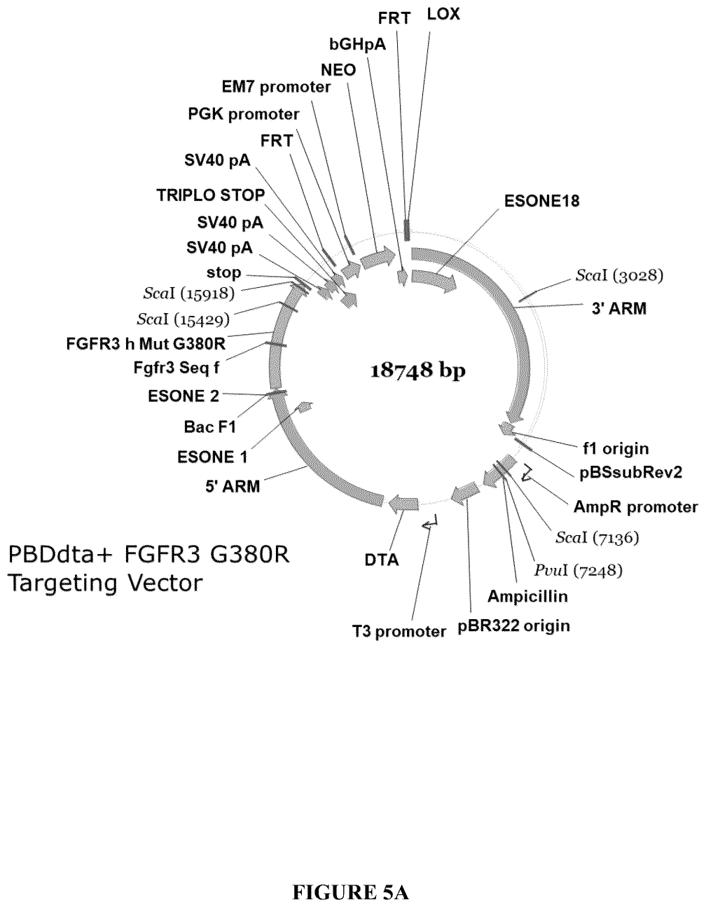

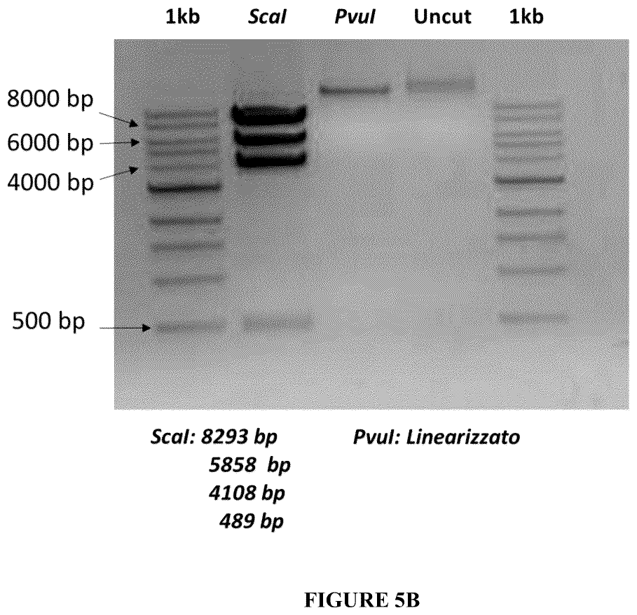

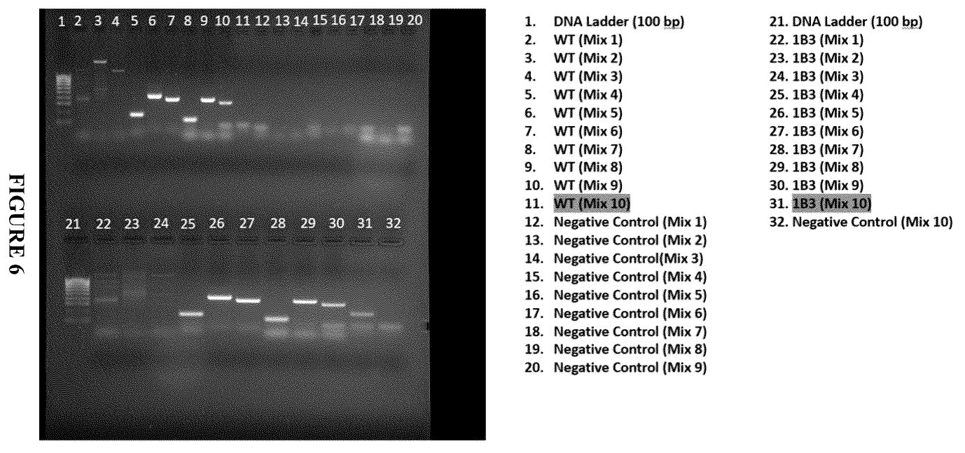

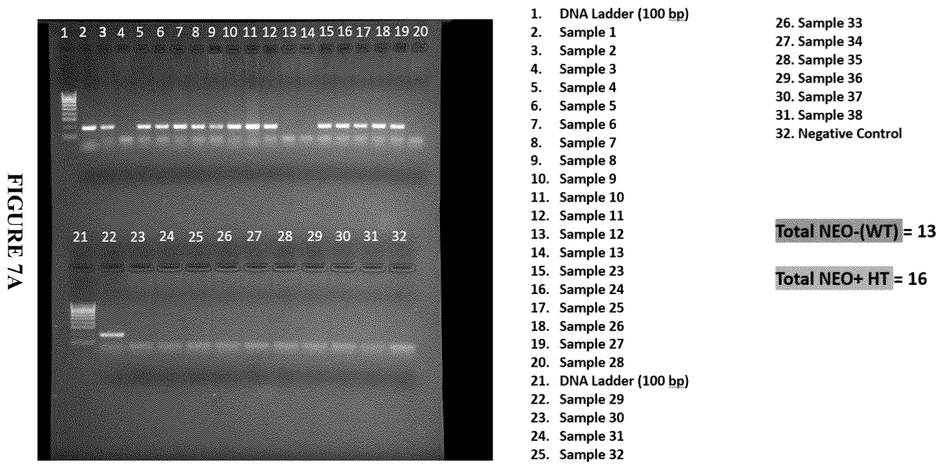

The targeting vector was designed by Biogem in collaboration with the University of L'Aquila (UNIVAQ). The vector was designed to replace the mouse Fgfr3 gene with the human FGFR3 coding DNA sequence carrying the G380R mutation, via homologous recombination (FIG. 5). The targeting vector was then used to transfect mouse embryonic stem cells (ESCs) via electroporation, and two ESC clones out of several hundred (approx. 300) were identified (Table 7) as useful for generating appropriate chimeras. These clones were therefore selected, injected into recipient blastocysts and implanted into foster mothers.

Chimaeras obtained from the ESC KI experiments were subjected to two different mating strategies to optimise the rate of success in obtaining the desired animal model:
Strategy 1: chimeras mating with wild-type (WT) animals, then with deleter FlpE animals (for removing the Neomycin cassette)·
Strategy 2: chimeras mating with deleter FlpE animals

All mice produced from these mating strategies were subjected to tail biopsy, and the relevant samples were genotyped to select heterozygous (HT) mice for generating a stable ACH colony.

Screening of ACH Primers on Genomic DNA Derived from ESC Clones and Genotyping of Mice Derived from Mating of Chimeras with WT Animals, then with FlpE (Strategy 1)

TABLE 7
ESC clones used for generating chimeras and
subsequent primer screening and genotyping.

| Sample of | | |
|---|---|---|
| ESC Clone | No. of cells | No. of cells |
| 1B 3 | 2 vials each containing | 2 vials each containing |
| | 500,000 cells | 1 × 10⁶ cells |
| 3C 9 | 2 vials each containing | 2 vials each containing |
| | 500,000 cells | 1 × 10⁶ cells |
| 22/S9 (WT) | 1 vial containing 1 × | |
| | 10⁶ cells | |

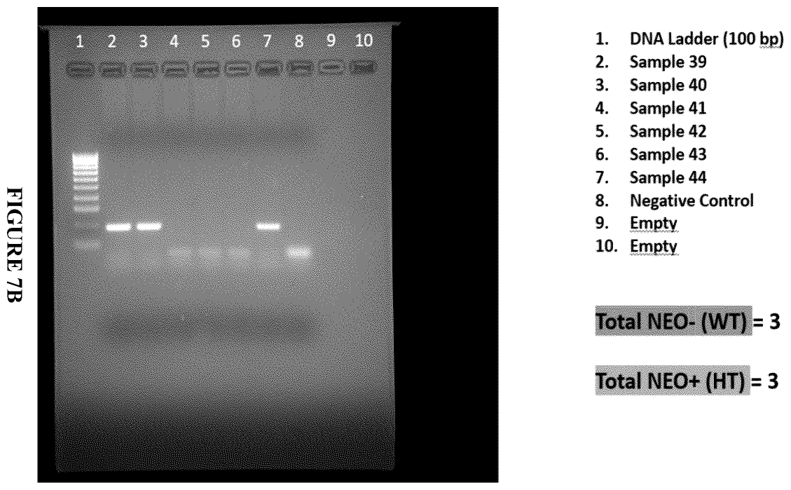

ES cell clones were digested and genomic DNA extracted. All extracted DNA samples were resuspended in an initial volume of 20 μL of TE Buffer and analyzed by Nanodrop to evaluate the concentration and purity of genomic DNA. One DNA sample derived from 1B3 ES cell clone and one sample derived from 22/S9 (WT) ES cell clone were selected and normalized (to obtain for all samples a DNA concentration of 100 ng/μL to use in the PCR system) for PCR analyses. Selected DNA samples were analyzed using 10 different reaction mixes, each containing a specific pair of primers to test (Table 8). Results are shown in FIG. 6. The actual sequences of all primers tested for identifying heterozygous animals in genotyping experiments are shown in Table 12 at the end of this Example.

TABLE 8
Primers used in reaction mixes for identifying heterozygous (HT) animals

| MIX 1 | MIX 2 | MIX 3 | MIX 4 | MIX 5 | MIX 6 | MIX 7 | MIX 8 | MIX 9 | MIX 10 |
|---|---|---|---|---|---|---|---|---|---|
| Primer FW: | Primer FW: | Primer FW: | Primer FW: | Primer FW: | Primer FW: | Primer FW: | Primer FW: | Primer FW: | Primer FW: |
| ACH LOXP | ACH LOXP | ACH LOXP | WT Geno 1 | WT Geno 1 | WT Geno 1 | WT Geno 2 | WT Geno 2 | WT Geno 2 | NEO ACH |
| Geno 1 | Geno 1 | Geno 1 | SEQ ID 63 | SEQ ID 63 | SEQ ID 63 | SEQ ID 64 | SEQ ID 64 | SEQ ID 64 | SEQ ID 69 |
| SEQ ID 68 | SEQ ID 68 | SEQ ID 68 | | | | | | | |
| Primer RV: | Primer RV: | Primer RV: | Primer RV: | Primer RV: | Primer RV: | Primer RV: | Primer RV: | Primer RV: | Primer RV: |
| ACH Geno 0 | ACH Geno 1 | ACH Geno 2 | ACH Geno 0 | ACH Geno 1 | ACH Geno 2 | ACH Geno 0 | ACH Geno 1 | ACH Geno 2 | NEO ACH |
| SEQ ID 60 | SEQ ID 61 | SEQ ID 62 | SEQ ID 60 | SEQ ID 61 | SEQ ID 62 | SEQ ID 60 | SEQ ID 61 | SEQ ID 62 | SEQ ID 70 |

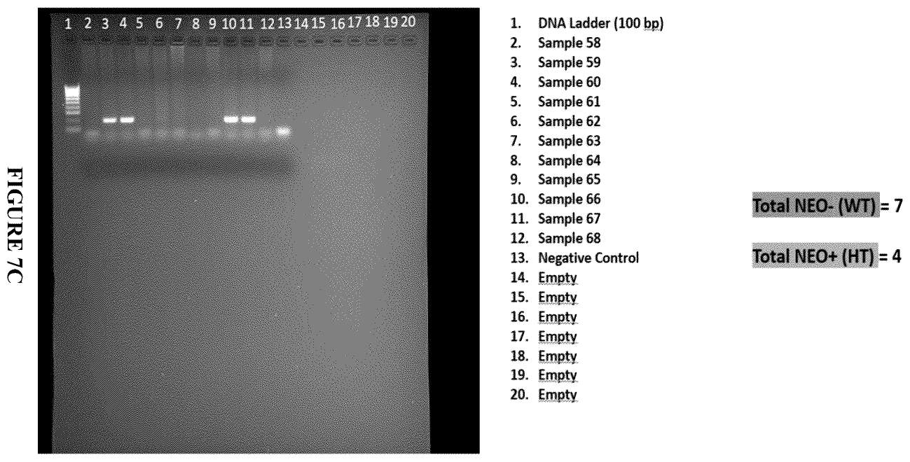

Table 9 shows the samples obtained from the progeny of crosses between chimaeras and WT animals. Analysis of the tail biopsy samples listed in Table 9, using primer mix 10 (in table 8), was able to discriminate between animals carrying the neomycin cassette (NEO+ HT, i.e. carrying the FGFR 3^{G380R} mutation) and neomycin negative (NEO− WT, i.e. not carrying the desired mutation) (FIG. 7). A total of 24 Neomycin positive and 33 Neomycin negative animals were identified. Neomycin positive animals were selected for further mating with FlpE mice and subsequent generation of a stable ACH colony. The primer sequences in primer mix 10 (in Table 8) were:

Forward NEO ACH:
[SEQ ID NO. 69]
CCTGCAGCCTGTTGACAATT
Reverse NEO ACH:
[SEQ ID NO. 70]
CATCAGAGCAGCCGATTGTC

TABLE 9
Litters and samples obtained from progeny
of crosses between chimeras and WT animals

| | | | | | |
|---|---|---|---|---|---|
| Litter | 1 I (1-13) | 3 I (23-30) | 7 I (31-37) | 8 I (38-44) | 6 I (58-60) |
| Birth | 7 Jan. 2023 | 7 Jan. 2023 | 10 Jan. 2023 | 8 Jan. 2023 | 19 Jan. 2023 |
| Mother | C57/B6 WT | C57/B6 WT | C57/B6 WT | C57/B6 WT | C57/B6 WT |
| Father | Chimaera 1 I | Chimaera 3 I | Chimaera 7 I | Chimaera 8 I | Chimaera 6 I |
| Sample | 1-5 (females | 23-24 (females | 31-34 (females | 38-39 (females | 58-59 (females |
| IDs | agouti) | agouti) | agouti) | agouti) | black) |
| | 6-9 (females | 25-26 (females | 35-37 (males | 40-41 (females | 60 (male |
| | black) | black) | black) | black) | agouti) |
| | 10 (male | 27-30 (males | | 42-43 (males | |
| | agouti) | black) | | agouti) | |
| | 11-13 (males | | | 44 (male | |
| | black) | | | black) | |

| | | | | |
|---|---|---|---|---|
| Litter | 20 I (61-68) | 14 L (70-79) | 11L (82-83) | 8 I (84-87) |
| Birth | 23 Jan. 2023 | 24 Jan. 2023 | 1 Feb. 2023 | 01 Feb. 2023 |
| Mother | C57/B6 WT | C57/B6 WT | C57/B6 WT | C57/B6 WT |
| Father | Chimaera 20 I | Chimaera 14 L | Chimaera 11 L | Chimaera 8 I |
| Sample | 61-63 (females | 70-72 (females | 82 (male | 84 (female |
| IDs | agouti) | agouti) | agouti) | agouti) |
| | 64-66 (females | 73-74 (females | 83 (male | 85 (male |
| | black) | black) | black) | black) |
| | 67-68 (males | 75-79 (males | | 86-87 (male |
| | black) | agouti) | | black) |

Genotyping of Mice Derived from Chimeras Crossed Directly with FlpE Animals (Strategy 2)

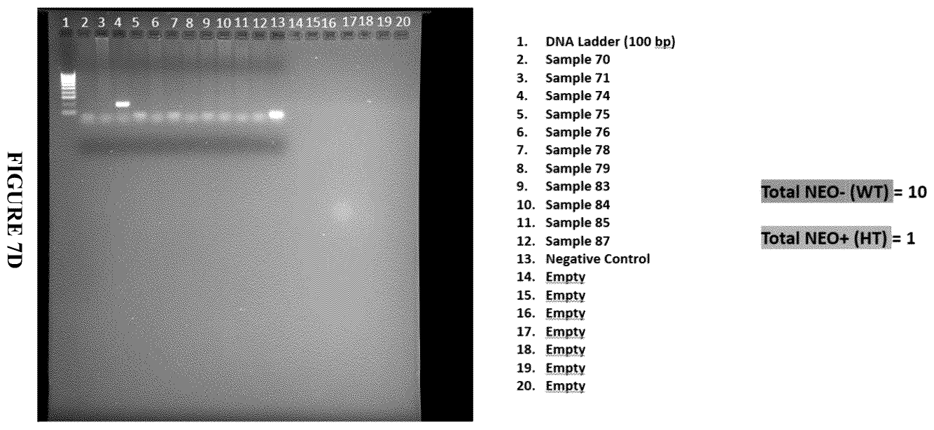

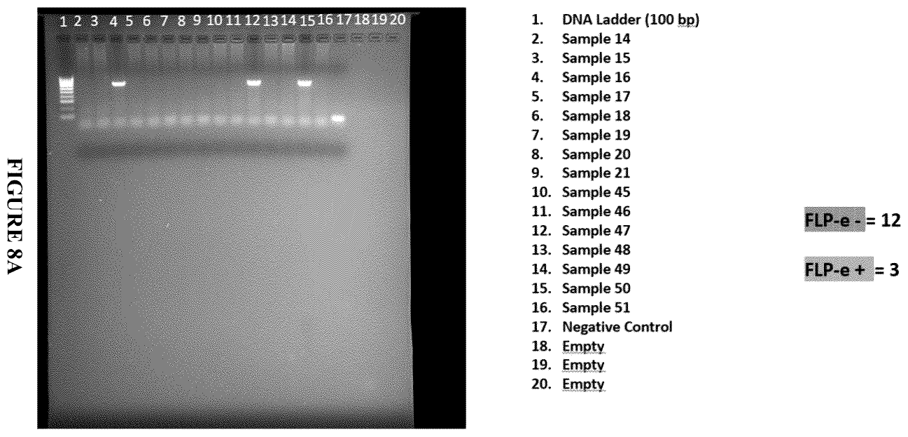

Biopsy samples were also analysed for pups generated in the second mating strategy of crossing chimaeras directly with FlpE mice (listed in Table 10). Analysis of the tail biopsy samples listed in Table 10, using primer mix 10, was able to discriminate between animals carrying FlpE (FLP-e+, i.e. considered as potential HT carrying the FGFR 3G380R mutation) and FlpE negative animals (FLP-e−, i.e. not carrying the desired mutation) (FIG. 8). A total of 9 FlpE positive and 24 FlpE negative animals were identified.

TABLE 10
Litters and samples obtained from progeny of crosses between chimeras and FlpE animals

| | | | | | | |
|---|---|---|---|---|---|---|
| Litter | 2 I (14-22) | 9 I (45-51) | 5 I (52-57) | 10 I (69) | 9 1 (80-81) | 2 I (88-95) |
| Birth | 10 Jan. 2023 | 10 Jan. 2023 | 17 Jan. 2023 | 24 Jan. 2023 | 1 Feb. 2023 | 2 Feb. 2023 |
| Mother | C57/B6 Flp-e | C57/B6 Flp-e | C57/B6 Flp-e | C57/B6 Flp-e | C57/B6 Flp-e | C57/B6 Flp-e |
| Father | Chimaera 2 I | Chimaera 9 I | Chimaera 5 I | Chimaera 10 I | Chimaera 9 I | Chimaera 2 I |
| Sample | 14-16 (females | 45 (female | 52-54 (females | 69 (male | 80 (male | 88-90 (females |
| IDs | agouti) | agouti) | black) | agouti) | agouti) | black) |
| | 17-19 (females | 46-48 (males | 55-57 (males | | 81 (male | 91-95 (males |
| | black) | agouti) | black) | | black) | agouti) |
| | 20-21 (males | 49-51 (males | | | | |
| | agouti) | black) | | | | |
| | 22 (male | | | | | |
| | black) | | | | | |

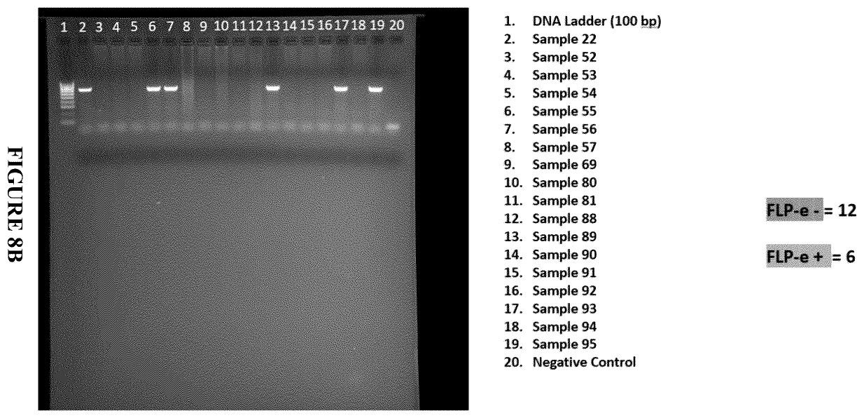

In order to further discriminate Flp-e mice, an additional PCR test was performed on the basis on the following primers listed in Table 11. DNA samples derived from biopsies of selected mice used for analysis were Sample 16 (FLP-e+ female Agouti) and Sample 22 (FLP-e+ male Black). Results are shown in FIG. 9. Primer mix 4 in Table 11 was selected for use in further analysis. Primer sequences for Primer Mix 4 in table 11 were:

Reverse ACH Geno 0:
[SEQ ID NO. 60]
GCTTGGTCTGTGGGACTGTT
Forward ACH MUT Geno 2:
[SEQ ID NO. 66]
ACGACTCCGTGTTTGCCCAC

TABLE 11
Primers tested for identifying HT
animals from second mating strategy

| | Primer forward | Primer reverse |
|---|---|---|
| Mix 1 | ACH MUT Geno 1 | ACH Geno 0 |
| | (SEQ ID NO. 65) | (SEQ ID NO. 60) |
| Mix 2 | ACH MUT Geno 1 | ACH Geno 1 |
| | (SEQ ID NO. 65) | (SEQ ID NO. 61) |
| Mix 3 | ACH MUT Geno 1 | ACH Geno 2 |
| | (SEQ ID NO. 65) | (SEQ ID NO. 62) |
| Mix 4 | ACH MUT Geno 2 | ACH Geno 0 |
| | (SEQ ID NO. 66) | (SEQ ID NO. 60) |
| Mix 5 | ACH MUT Geno 2 | ACH Geno 1 |
| | (SEQ ID NO. 66) | (SEQ ID NO. 61) |
| Mix 6 | ACH MUT Geno 2 | ACH Geno 2 |
| | (SEQ ID NO. 66) | (SEQ ID NO. 62) |
| Mix 7 | FRT ACH Geno 1 | ACH Geno 0 |
| | (SEQ ID NO. 67) | (SEQ ID NO. 60) |
| Mix 8 | FRT ACH Geno 1 | ACH Geno 1 |
| | (SEQ ID NO. 67) | (SEQ ID NO. 61) |
| Mix 9 | FRT ACH Geno 1 | ACH Geno 2 |
| | (SEQ ID NO. 67) | (SEQ ID NO. 62) |

TABLE 12
Sequences of all primers tested for identifying heterozygous
(HT) animals in genotyping experiments

| Name | Sequence | Seq ID No. |
|---|---|---|
| Reverse ACH Geno 0 | GCTTGGTCTGTGGGACTGTT | 60 |
| Reverse ACH Geno 1 | GGGAAATGAGAGGGCCAGAA | 61 |
| Reverse ACH Geno 2 | ACATTCTTGTTTGGTGCTCC | 62 |
| Forward ACH WT Geno 1 | GACCTCTCCGTGCCGTTTGA | 63 |
| Forward ACH WT Geno 2 | GCTCCAGCTCGTCCGGAGAT | 64 |
| Forward ACH MUT Geno 1 | TGTTTGCCCACGACCTGCTG | 65 |
| Forward ACH MUT Geno 2 | ACGACTCCGTGTTTGCCCAC | 66 |
| Forward ACH FRT Geno 1 | GTTCCTATTCTCTAGAAAGT | 67 |
| Forward ACH LOXP Geno 1 | CGTATAATGTATGCTATACG | 68 |
| Forward NEO ACH | CCTGCAGCCTGTTGACAATT | 69 |
| Reverse NEO ACH | CATCAGAGCAGCCGATTGTC | 70 |

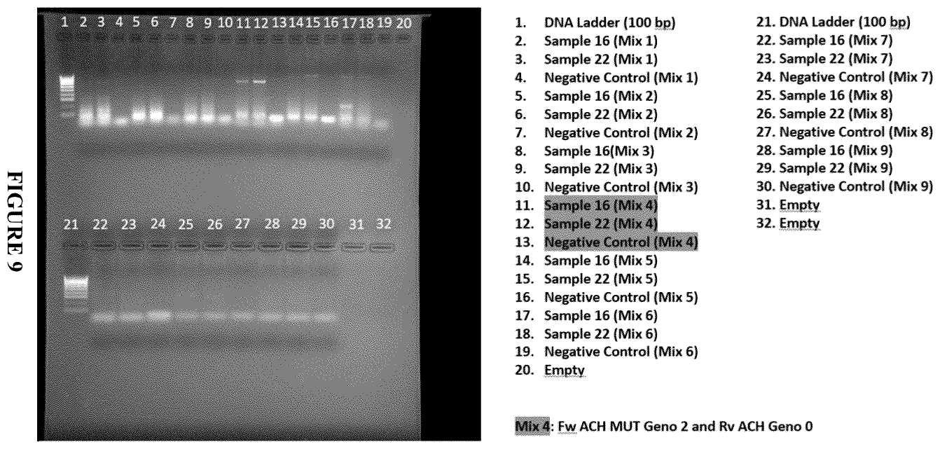

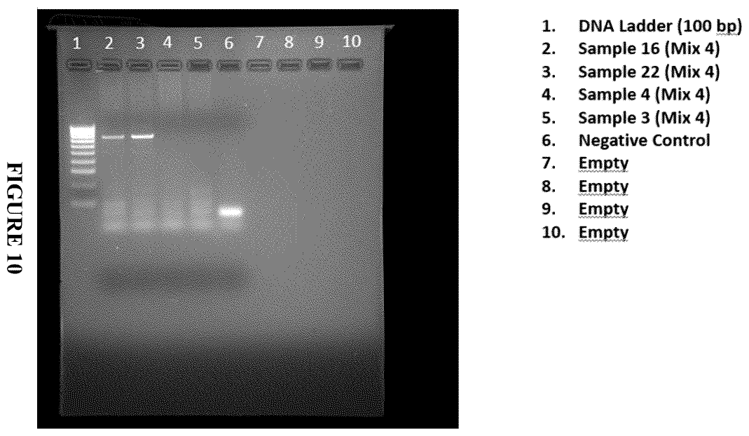

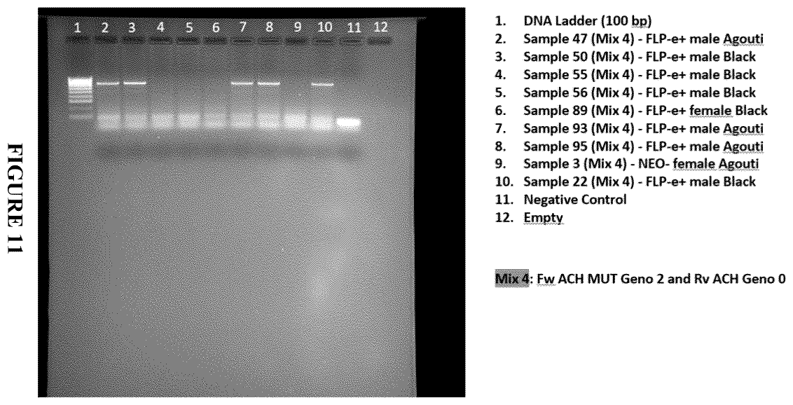

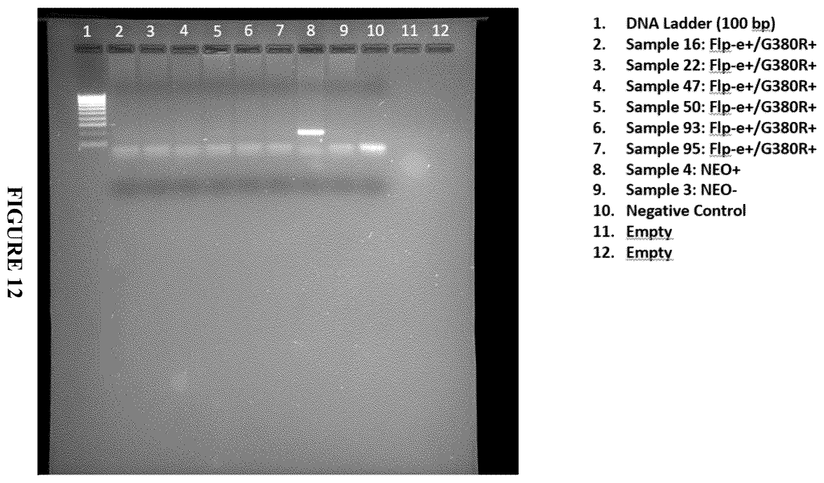

A further step, aiming to identify Neomycin cassette-free HT animals, tested two further samples (Sample 4: NEO+ female Agouti; Sample 3: NEO-female Agouti). Results for these compared with Samples 16 and 22 are shown in FIG. 10. As can be seen from the presence of the higher molecular weight band observed in samples 16 and 22, samples 16 and 22 both contained the G380R mutation, while samples 3 and 4 did not. Additional samples (47, 50, 55, 56, 89, 93, 95 as well as samples 3 and 22 as comparators) were also tested, using primer mix 4, and results are shown in FIG. 11. Samples 47, 50, 93 and 95 contained the G380R mutation. All G380R positive samples analysed (16, 22, 47, 50, 93 and 95) were then tested for the presence of the neomycin cassette, using sample 4 (NEO+) and sample 3 (NEO−) as reference samples, and found to be neomycin negative (FIG. 12).

Additional testing of samples obtained from the second FlpE mating strategy identifies HT neomycin-free individuals that are also positive for ACH pathology. Animals from this second pool are integrated into the current ACH colony originally derived from the first mating strategy.

### Example 7: In Vivo Short-Term Screening of siRNA Formulations and Assessment of Biodistribution in Femur Growth Plate and Visceral Organs

Briefly, the biodistribution study is performed using a Cy5-tagged siRNA identified by the in vitro screen described herein combined with a lipid particle comprising hydrolysable silicon to form a complex as described elsewhere herein. Five day-old or three month-old C57BL/6 male wild-type mice (6 mice/group) are injected via the intraperitoneal (i.p.) route with the selected siRNA/SiS complex. A control group receives empty SiS-biocourier. Mice are sacrificed at 0 min, 30 min, 1 h, 6 h, 24 h and 48 h post-injection and their organs harvested, to assess the distribution of the complex by analysing the Cy5 fluorescence in different organs. Organ extracts are analysed by fluorometry and organ cryosections are analysed by confocal microscopy. siRNA molecules are also directly detected by stem-loop RT-PCR.

Pharmacokinetic studies are performed using catheterised 3 month-old C57BL/6 male wild-type mice (6 mice/group), administered 1 single injection of a fluorescent siRNA/lipid/Si complex as described in the biodistribution study above. Blood is collected through the jugular cannula in live mice, with time points of blood sampling selected according to the moderate elimination scheme (with a predicted t_{1/2} of below approximately 600 minutes). Sampling takes place at t=0, 15 minutes, 30 minutes, 1 hour, 2 hours, 6 hours, 24 hours, and 48 hours post-injection. Fluorescence levels in serum are evaluated using fluorometry and siRNA direct detection in serum is performed using stem-loop RT-PCR.

For toxicity investigation, both maximum tolerated dose (single dose, ascending) and dose range finding studies (single dose, repeated) are performed. These studies use 3 month-old C57BL/6 male wild-type mice (6 mice/group), treated with either an siRNA/lipid/Si complex as described in the biodistribution study above (experimental) or with 0.9% w/v NaCl solution (control). Blood collection is performed through the jugular cannula. The evaluations performed are:
Daily clinical observation;
Measurement of body weight (at each change of dose level);
Food consumption analysis (at each change of dose level);
Clinical pathology at sacrifice by haematology, blood chemistry and inflammatory cytokine ELISA assays;
Post-mortem evaluation: organ weights and macroscopic features;
Post-mortem evaluation: tissue preservation and target organ histopathology (liver, heart, lung, spleen, kidney, bone, muscle, and brain.

Whilst the present invention has been described and illustrated with reference to particular embodiments, it will be appreciated by those of ordinary skill in the art that the invention lends itself to many different variations not specifically illustrated herein.

Where in the foregoing description, integers or elements are mentioned which have known, obvious or foreseeable equivalents, then such equivalents are herein incorporated as if individually set forth. Reference should be made to the claims for determining the true scope of the present invention, which should be construed so as to encompass any such equivalents. It will also be appreciated by the reader that integers or features of the invention that are described as preferable, advantageous, convenient or the like are optional and do not limit the scope of the independent claims. Moreover, it is to be understood that such optional integers or features, whilst of possible benefit in some embodiments of the invention, may not be desirable, and may therefore be absent, in other embodiments.

1.-53. (canceled)

54. An siRNA molecule which comprises at least 17, for example, at least 18 or at least 19, consecutive nucleotides selected from the sequence GCAGGCAUCCUCAGCUACX_{M}GGGUGGGCUUCUUCCUGU (SEQ ID NO: 1), wherein X_{M} is selected from the nucleotides A and C, wherein said consecutive nucleotides selected from SEQ ID NO: 1 must include the X_{M} nucleotide which is found at position 19 of SEQ ID NO: 1; or a variant of said siRNA molecule having at least 1 and no more than 6 nucleotide substitutions with respect to SEQ ID NO: 1, provided that the X_{M} nucleotide at the position of said variant siRNA molecule which corresponds to position 19 of SEQ ID NO: 1 is not substituted and is thus selected from the nucleotides A and C, and wherein said siRNA molecule is not an siRNA of length 19 nucleotides having the sequence GAAGGCAUCCUCAGCUACA (SEQ ID NO: 71).

55. An siRNA molecule or variant thereof as claimed in claim 54, wherein the maximum length of the siRNA molecule or variant thereof is 30 or fewer nucleotides.

56. An siRNA molecule or variant thereof as claimed in claim 54, wherein the variant has at least 1 and no more than 3-nucleotide substitutions, with respect to SEQ ID NO: 1, provided that the X_{M} nucleotide at the position of said variant siRNA molecule which corresponds to position 19 of SEQ ID NO: 1 is not substituted and is thus selected from the nucleotides A and C.

57. An siRNA molecule or variant thereof as claimed in claim 54, which is at least partially double-stranded along the length of the sequence that is complementary to a target mRNA.

58. An siRNA molecule or variant thereof as claimed in claim 54, wherein the length of the siRNA molecule or variant thereof is at least 19 nucleotides and wherein the A of the siRNA molecule or variant thereof which corresponds to position 19 of SEQ ID NO: 1 may be found at any of the positions of the siRNA molecule or variant thereof.

59. An siRNA molecule or variant thereof as claimed in claim 54, which comprises a sequence selected from the group of sequences defined by the consensus sequence X₁X₂UAX₃X_{M}GGGUGGX₄CX₅UCUU (SEQ ID NO: 2), wherein X_{M} is selected from the group consisting of A and C and wherein X₁, X₂, X₃, X₄ and X₅ are each independently selected from the group consisting of A, G, C and U.

60. An siRNA molecule or variant thereof as claimed in claim 54, which comprises a sequence selected from the group of sequences defined by the consensus sequence X_{M}X₁X₂GUGGGCUUCX₃UX₄CUGU (SEQ ID NO: 5), wherein X_{M} is selected from the group consisting of A and C and wherein X₁, X₂, X₃ and X₄ are each independently selected from the group consisting of A, G, C and U.

61. An siRNA molecule or variant thereof as claimed in claim 54, which comprises a sequence selected from the group of sequences defined by the consensus sequence X₁CAGCUAX₂X_{M}X₃GGX₄X₅GGCUU (SEQ ID NO: 8), wherein X_{M} is selected from the group consisting of A and C and wherein X₁, X₂, X₃, X₄ and X₅ are each independently selected from the group consisting of A, G, C and U.

62. An siRNA molecule or variant thereof as claimed in claim 54, which comprises a sequence selected from the group of sequences defined by the consensus sequence X₁CAUCCUCAGCX₂X₃X₄X_{M}X₅GX₆U (SEQ ID NO: 11), wherein X_{M} is selected from the group consisting of A and C and wherein X₁, X₂, X₃, X₄, X₅ and X₆ are each independently selected from the group consisting of A, G, C and U.

63. An siRNA molecule or variant thereof as claimed in claim 54, which comprises a sequence selected from the group of sequences defined by the consensus sequence GX₁AGGX₂AUCCUCX₃GX₄UAX₅X_{M} (SEQ ID NO: 13), wherein X_{M} is selected from the group consisting of A and C and wherein X₁, X₂, X₃, X₄ and X₅ are each independently selected from the group consisting of A, G, C and U.

64. An siRNA molecule or variant thereof as claimed in claim 54, wherein X_{M} is A.

65. An siRNA molecule as claimed in claim 54 which comprises a sequence selected from the group consisting of:

| | Sequence |
|---|---|
| | (Bold nucleotides indicate G1138A |
| | mutation; Underlined nucleotides |
| | indicate additional mismatches |
| | relative to the human wild-type |
| SEQ ID NO. | FGFR3 sequence) |
| 4 | GCUACAGGGUGGGCAUCUU |
| 3 | GCUAUAGGGUGGGCUUCUU |
| 15 | GCAGGCAUCCUCAGCUACA |
| 16 | GCAGGCAUCCUCAGCUAUA |
| 17 | GCAGGCAUCCUCAGGUACA |
| 18 | GCAGGCAUCCUCUGCUACA |
| 19 | GCUACAGGGUGGGCUUCUU |
| 20 | GCUACAGGGUGGCCUUCUU |
| 21 | AGGGUGGGCUUCUUCCUGU |
| 6 | AAGGUGGGCUUCUUCCUGU |
| 7 | AGGGUGGGCUUCUUUCUGU |
| 22 | AGGGUGGGCUUCAUCCUGU |
| 23 | UCAGCUACAGGGUGGGCUU |
| 24 | UCAGCUAUAGGGUGGGCUU |
| 9 | UCAGCUACAGGGUCGGCUU |
| 10 | UCAGCUACAGGGAGGGCUU |
| 25 | GCAUCCUCAGCUACAGGGU |
| 12 | GCAUCCUCAGCUAUAGGGU |
| 26 | GCAUCCUCAGCUUCAGGGU |
| 27 | GCAUCCUCAGCCACAGGGU |
| 28 | GUAGGCAUCCUCAGCUACA |
| 29 | GCAGGUAUCCUCAGCUACA |
| 30 | GUUACAGGGUGGGCUUCUU |
| 31 | UCUACAGGGUGGGCUUCUU |
| 32 | AGUGUGGGCUUCUUCCUGU |
| 33 | UCAUCCUCAGCUACAGGGU |
| 34 | GCAUCCUCAGCUACAUGGU |
| 35 | GCAUCCUCAGCUACAGGAU |
| 36 | ACAGCUACAGGGUGGGCUU |
| 37 | UCAGCUACAUGGUGGGCUU |

66. An siRNA molecule as claimed in claim 54 which is selected from the group consisting of GCUAUAGGGUGGGCUUCUU (SEQ ID NO: 3), GCUACAGGGUGGGCAUCUU (SEQ ID NO: 4), AAGGUGGGCUUCUUCCUGU (SEQ ID NO: 6), AGGGUGGGCUUCUUUCUGU (SEQ ID NO: 7), UCAGCUACAGGGUCGGCUU (SEQ ID NO: 9), UCAGCUACAGGGAGGGCUU (SEQ ID NO: 10) and GCAUCCUCAGCUAUAGGGU (SEQ ID NO: 12).

67. An siRNA molecule which comprises at least 17 consecutive nucleotides selected from the sequence GX₁AGX₂X₃AUCCX₄CX₅X₆X₇UX₈X₉X_{M}X₁₀X₁₁X₁₂X₁₃X₁₄GX₁₅CX₁₆UCX₁₇UX₁₈CUGU (SEQ ID NO: 14), wherein X_{M} is selected from the nucleotides A and C, wherein said consecutive nucleotides selected from SEQ ID NO: 14 must include the X_{M} nucleotide which is found at position 19 of SEQ ID NO: 14; wherein X₁ is selected from C and U, X₂ is selected from G and U, X₃ is selected from C and U, X₄ is selected from A and U, X₅ is selected from A and U, X₆ is selected from G and U, X₇ is selected from C, G and U, X₈ is selected from A and U, X₉ is selected from C and U, X₁₀ is selected from G, A and U, X₁₁ is selected from G and U, X₁₂ is selected from G and A, X₁₃ is selected from A and U, X₁₄ is selected from G and C, X₁₅ is selected from G and C, X₁₆ is selected from A and U, X₁₇ is selected from A and U, and X₁₈ is selected from C and U.

68. A method of manufacturing an siRNA molecule or variant thereof as claimed in claim 54 comprising the step of synthesising said siRNA molecule or variant thereof.

69. A pharmaceutical composition comprising an siRNA molecule or variant thereof as claimed in claim 54, and a pharmaceutically acceptable carrier.

70. A complex comprising:
(i) a particle comprising hydrolysable silicon; and
(ii) an siRNA or variant thereof as claimed in claim 54 which is associated with said particle.

71. A complex comprising:
(i) a particle comprising hydrolysable silicon;
(ii) an siRNA which targets an mRNA encoding a mutant FGFR3 protein,
wherein said siRNA targets a portion of the mRNA sequence which includes nucleotides encoding a Gly to Arg mutation at position 380 of the protein, and wherein said siRNA is associated with said particle.

72. A method of treating achondroplasia in a subject in need thereof, comprising administering to the subject an siRNA molecule or variant thereof as claimed in claim 54.

73. A method of downregulating the expression of FGFR3 protein carrying a Gly to Arg mutation at position 380 (G380R) in a subject in need thereof, comprising administering an siRNA molecule or variant thereof as claimed in any of claim 54.
